(19) United States
(12) Patent Application Publication
(10) Pub. No.: US 2023/0248881 A1
(43) Pub. Date: Aug. 10, 2023

(54) IMPLANTS FOR SCULPTING, AUGMENTING OR CORRECTING FACIAL FEATURES SUCH AS THE CHIN

(71) Applicant: Allergan Industrie, SAS, Pringy (FR)

(72) Inventors: Jean-Xavier ROCA MARTINEZ, Annecy (FR); Aurore AYGLON, Cruseilles (FR)

(21) Appl. No.: 17/943,092

(22) Filed: Sep. 12, 2022

Related U.S. Application Data
(63) Continuation of application No. 16/370,679, filed on Mar. 29, 2019, now abandoned.
Application No. 16/370,679 is a continuation of application No. 16/351,400, filed on Mar. 12, 2019, now abandoned.
Application No. 16/351,400 is a continuation-in-part of application No. 15/550,763, filed on Aug. 11, 2017, now abandoned.

(30) Foreign Application Priority Data
Feb. 13, 2015 (WO) PCT/FR2015/050357
Feb. 16, 2015 (WO) PCT/IB2015/000350

(51) Int. Cl. A61L 27/20; A61L 27/12; A61L 27/52; C08B 37/08; A61L 27/54; C08L 5/08

(52) U.S. Cl. CPC A61L 27/20; A61L 27/12; A61L 27/52; C08B 37/0072; A61L 27/54; C08L 5/08; A61L 2400/06; A61M 5/329

(57) ABSTRACT

Methods of treating chin retrusion in a subject by increasing a glabella-subnasale-pogonion (G-Sn-Pog) facial angle of the subject are provided, which may include injecting a first volume of a dermal filler into a pogonion, injecting a second volume of a dermal filler into a mentum, and/or injecting a third volume of the dermal filler into a left pre-jowl sulcus and/or a right pre-jowl sulcus, wherein the G-Sn-Pog facial angle of the subject is increased by at least about 1° or more. Methods of treating chin retrusion are provided that may include injecting a first volume of a dermal filler into a pogonion, injecting a second volume of a dermal filler into a mentum, and/or injecting a third volume of the dermal filler into a left pre-jowl sulcus and/or a right pre jowl sulcus, wherein the chin retrusion of the subject is decreased by at least about 2 mm.

### CROSS-REFERENCE TO RELATED APPLICATIONS

This application is a continuation of Ser. No. 16/370,679, filed on Mar. 29, 2019, which is a continuation-in-part of U.S. patent application Ser. No. 16/351,400, filed on Mar. 12, 2019, which is a continuation-in-part of U.S. patent application Ser. No. 15/550,763, filed on Aug. 11, 2017, which is the National Phase application of International Application No. PCT/EP2016/053009, filed Feb. 12, 2016, which claims priority to International Application No. PCT/FR2015/050357, filed Feb. 13, 2015, and to International Application No. PCT/IB2015/000350, filed Feb. 16, 2015, each of which is incorporated herein by reference in their entirety.

### BACKGROUND

The present disclosure generally relates to injectable compositions and more specifically relates to injectable implants for adding structure and contour to the lower face.

Dermal fillers are injectable, biocompatible compositions which are well known to correct wrinkles and folds and add volume to the face. Hyaluronic acid (HA) is still considered by many to be one of the most desirable dermal fillers in that it does not pose the risk of an allergic reaction and it is temporary and reversible. The great majority of hyaluronic acid-based dermal fillers have been specifically developed for treating wrinkles and folds in skin. To be useful for facial contouring or substantial volumizing, it would be advantageous to increase the bulking effect of the compositions, also referred to as “lift.” It would also be advantageous to maximize resistances of the compositions to shear and normal deformation happening in the soft tissues of the face. One of the drawbacks of maximizing these resistances, for example, elasticity and cohesivity, is that it is expected that in doing so, the viscosity of the compositions will increase to the point that they become difficult to inject with a thin needle.

There is therefore a great need for an injectable HA based implant that is specifically designed to be effective in adding substantial volume to the face, for example, for contouring the lower face, for example, for augmenting or correcting the chin, for example, for correction of chin retrusion, or for example, for augmenting or correcting the nose. It would be highly advantageous if such an implant, despite, its high viscosity, would remain easy to inject with a thin needle.

The shape of the chin has long been recognized as an important feature of the face that elicits a strong aesthetic perception that tends to be associated with personality traits of an individual. A deficient chin that lacks projection is commonly labeled a “weak chin” while prominent chins are labeled “strong chins,” both implying strength of personality.

Several studies have suggested that faces with average proportions are viewed as the most attractive and that small features including a small chin are interpreted as attractive in females while the expanded chin and jaw, as a result of maturation, are interpreted as attractive in males. The appearance of the chin is a determinant of perceived attractiveness and can even influence an individual's psychosocial well-being.

Chin augmentation is conventionally performed by surgically placing a permanent implant above the jaw. The procedure is currently among the top aesthetic surgical procedures performed, based on the American Society for Aesthetic Plastic Surgery (ASAPS), and has increased 71% since 2010.

A retrusive chin can be the result of changes in growth of the lower third of the face during maturation, trauma, or facial aging, the latter of which may exacerbate the deformities or asymmetries caused by the former two. The shape of the mandible affects the mouth, chin, and neck. As an individual ages, the reduction in skeletal support of this region makes soft tissue atrophy prominent, exaggerating jowls, decreasing chin protrusion, and making the jawline look weak. Chin deformities are among the most common bony abnormalities of the face, the most common of which is horizontal microgenia characterized by the presence of normal vertical height with a retruded bony chin.

As the mandible and chin make up the framework of the lower face, augmentation methods to treat age-related chin retrusion and contour changes of the chin area or to treat microgenia have been explored for decades. Where the approach in correcting chin retrusion is to add volume, treatment methods have included chin implants, genioplasty, and injection of silicone and semi-permanent fillers, such as polymethylmethacrylate microspheres, and calcium hydroxyapatite. However, all of these treatment methods have drawbacks. For example, chin implants and genioplasty involve painful surgery that may not result in correction of chin retrusion and aesthetic blending of the area. This approach may exacerbate bone resorption and infection, resulting in the need for implant removal. Injection of semi-permanent fillers have trade-offs between volumizing capacity and adverse events associated with semi-permanent fillers.

### SUMMARY

Accordingly, an injectable implant is provided for facial sculpturing, for example, for augmenting, correcting, restoring or creating volume in the chin and other facial features in a human being. In some embodiments, the injectable implant increases a glabella-subnasal-pogonion (G-Sn-Pog) facial angle in a subject in need thereof and/or increases chin protrusion in a subject in need thereof.

The present disclosure provides temporary, reversible, HA-based structural gels manufactured specifically to provide a safe, minimally invasive method to create facial volume or facial contours. The present implants provide improved volumizing and lift properties relative to other HA-based injectables, due to a combination of mechanical properties including high elasticity and high cohesivity, while still being easily injectable with a thin needle. The present implants may be used for injection into the subcutaneous and/or supraperiosteal space. In many embodiments the implants are moldable after injection, and therefore permit sculpting, contouring, and shaping across the injected areas, for example, the chin and jaw area. In some embodiments, the dermal implants are dermal fillers.

The implants generally comprise a composition comprising a hyaluronic acid (HA) crosslinked with a crosslinking agent selected from the group consisting of 1,4-butanediol diglycidyl ether (BDDE), 1,4-bis(2,3-epoxypropoxy)butane, 1,4-bisglycidyloxybutane, 1,2-bis(2,3-epoxypropoxy)ethylene and 1-(2,3-epoxypropyl)-2,3-epoxycyclohexanethe. In some embodiments, the implants generally comprise a composition comprising a hyaluronic acid (HA) crosslinked with BDDE. The compositions are suitable for injection, for example, through a fine gauge needle, and are capable of augmenting, correcting, or creating volume or lift in the face, for example, the lower face, for example, the chin or jaw, or for the midface, for example, the nose.

In some embodiments of each or any of the above or below mentioned embodiments, the HA concentration is greater than 20 mg/g. In some embodiments, the HA concentration is about 21 mg/g, or about 22 mg/g, or about 23 mg/g, or about 24 mg/g, or about 25 mg/g, or about 26 mg/g, or about 27 mg/g, or about 28 mg/g, or about 29 mg/g, or about 30 mg/g or greater. In other embodiments, the composition has an HA concentration of between 22.5 mg/g to 27.5 mg/g, for example, 25.0 mg/g.

In some embodiments of each or any of the above or below mentioned embodiments, the method adds volume and lift to the chin or jawline or nose of the patient for a period of time in the range of about 9 months to about 24 months after the administration or injection into the chin or jawline of the patient. The composition may be moldable, for example, by physical manipulation of the tissue near the implant for a period of time after injection. The compositions may have a setting time, when the composition is no longer moldable and substantially retains its shape for the duration of the implant, within about 24 to about 48 hours after being implanted or injected.

In some embodiments of each or any of the above or below mentioned embodiments, the compositions further include an anesthetic agent, for example, lidocaine HCl. For example, the compositions may include about 0.3% w/w lidocaine HCl.

In preferred embodiments of each or any of the above or below mentioned embodiments, the compositions comprise a hyaluronic acid gel, preferably in an amount of about 25 mg; and lidocaine hydrochloride, preferably in an amount of about 3 mg, in a phosphate buffer (pH 7.2), preferably in a volume q.s. 1 mL.

In some embodiments of each or any of the above or below mentioned embodiments, compositions are made with a mixture of low molecular weight hyaluronic acid and high molecular weight hyaluronic acid. For example, the crosslinked hyaluronic acid may be made from about 50% and about 100% of a low molecular weight hyaluronic acid prior to being crosslinked with the crosslinking agent. In some embodiments, the crosslinked hyaluronic acid is made from about 70% to about 90% of a low molecular weight hyaluronic acid prior to being crosslinked with the crosslinking agent. In some embodiments, the crosslinked hyaluronic acid is made from about 90% of a low molecular weight hyaluronic acid prior to being crosslinked with the crosslinking agent.

Using primarily a low molecular weight HA prior to crosslinking, for example about 50% or greater, for example, about 70% or about 90% low molecular weight HA, rather than using primarily a high molecular weight HA, produces a more robust, longer lasting, moldable hydrogel, having a higher cohesivity and elasticity, and more specifically suitable for facial sculpturing and augmentation by means of subcutaneous or supraperiosteal injection.

In some embodiments of each or any of the above or below mentioned embodiments, the HA has a degree of crosslinking of between about 4% and about 12%. For example, the HA has a degree of crosslinking of about 4%, or about 6%, or about 8%, or about 10%. In some embodiments the HA has a degree of crosslinking of about 6.5%. In other embodiments, the HA has a degree of crosslinking of about 7.5%, or about 8.5%, or about 9.5%, or about 10.5%.

In another aspect of the disclosure, methods for correcting chin retrusion in a patient are provided. The methods may generally comprise supraperiostally administering in the chin of the patient, an effective amount of a composition comprising BDDE-crosslinked hyaluronic acid (HA), the HA having a degree of crosslinking of about 10%, and having a HA concentration of greater than 20 mg/g. In a preferred embodiment, the HA concentration is about 25 mg/g.

In a specific embodiment, the compositions comprise low molecular weight hyaluronic acid (NaHA) crosslinked with about 10% BDDE (w/w), and formulated to a concentration of about 25 mg/g with 0.3% lidocaine hydrochloride (w/w) in a phosphate buffer, pH 7.2, and supplied in a 1 mL COC (cyclic olefin copolymer) syringe.

In some embodiments of each or any of the above or below mentioned embodiments, the compositions are extrudable through a fine gauge needle, for example, a needle having a gauge of 25 G, 26 G, 27 G, 28 G, 29 G or 30 G. In a specific embodiment, the needle is a needle of 27 gauge×13 mm/27 G½×26 mm.

An extrusion force is the force (in Newtons N) needed to extrude the composition from its syringe at a certain rate. For example, with the supplied 1 mL COC syringe and a TSK 27 G×13 mm needle, the extrusion force of some of the compositions of this disclosure can be between about 4N and about 15N at 13 mm/min, which is considered as very low. In some embodiments of each or any of the above or below mentioned embodiments, the extrusion force can be between about 7N and about 12N, and preferably between about 8N and about 10 N.

In another aspect of the disclosure, methods are provided for contouring or correcting a facial feature, for example, a retruded chin, of an individual. The methods may comprise, for example, the step of subdermally administering into a treatment area of the patient, an effective amount, for example, about 1.0 ml, or more, for example, about 2.0 ml or more, for example, about 3.0 ml or more, for example, 4.0 mL, of a composition of the disclosure. The facial feature to be improved or contoured may be a chin, for example, a retruded chin of a patient. The treatment area may include an area selected from the group consisting of the pogonion, the mentum, the left pre jowl sulcus, the right pre jowl sulcus, and the sublabial crease. The treatment may comprise administering the composition into two or more of the treatment areas.

In another aspect of the disclosure, methods of increasing a G-Sn-Pog facial angle in a subject in need thereof are provided. The methods may generally comprise injecting a first volume of a composition comprising a dermal filler into a pogonion, injecting a second volume of the dermal filler into a mentum, and injecting a third volume of the dermal filler into a left pre jowl sulcus and a right pre jowl sulcus, wherein the G-Sn-Pog facial angle of the subject is increased after injection of the dermal filler into the pogonion, mentum, left pre-jowl sulcus, and right pre jowl sulcus by at least 1°.

In some embodiments of each or any of the above or below mentioned embodiments, the increase in the G-Sn-Pog facial angle of the subject is maintained for at least about 1 month, about 2 months, about 3 months, about 4 months, about 5 months, about 6 months, about 7 months, about 8 months, about 9 months, about 10 months, about 11 months, about 12 months, about 13 months, about 14 months, about 15 months, about 16 months, about 17 months, about 18 months, or more.

In some embodiments of each or any of the above or below mentioned embodiments, the increase in the G-Sn-Pog facial angle of the subject is increased by at least about 0.5°, about 0.6°, about 0.7°, about 0.8°, about 0.9°, about 1°, about 1.1°, about 1.2°, about 1.3°, about 1.4°, about 1.5°, about 1.6°, about 1.7°, about 1.8°, about 1.9°, about 2°, about 2.1°, about 2.2°, about 2.3°, about 2.4°, about 2.5°, about 2.6°, about 2.7°, about 2.8°, about 2.9°, about 3°, about 3.1°, about 3.2°, about 3.3°, about 3.4°, about 3.5°, about 3.6°, about 3.7°, about 3.8°, about 3.9°, about 4°, about 4.1°, about 4.2°, about 4.3°, about 4.4°, about 4.5°, about 4.6°, about 4.7°, about 4.8°, about 4.9°, about 5°, about 5.1°, about 5.2°, about 5.3°, about 5.4°, about 5.5°, about 5.6°, about 5.7°, about 5.8°, about 5.9°, about 6.0°, or more.

In some embodiments of each or any of the above or below mentioned embodiments, the injections into the pogonion, mentum, left pre-jowl sulcus, and right pre-jowl sulcus comprise a serial puncture technique. In some embodiments, the injections into the pogonion, mentum, left pre-jowl sulcus, and right pre-jowl sulcus comprise a supraperiosteal injection.

In some embodiments of each or any of the above or below mentioned embodiments, the method further comprises injecting a fourth volume of the dermal filler into a sublabial crease. In some embodiments, the injection into the sublabial crease comprises a subcutaneous injection.

In some embodiments of each or any of the above or below mentioned embodiments, the total volume of the dermal filler injected into the pogonion, mentum, left pre-jowl sulcus, right pre-jowl sulcus, and sublabial crease is no greater than 3.0 mL, 4.0 mL, or 5.0 mL.

In some embodiments of each or any of the above or below mentioned embodiments, each injection volume is no greater than 2.0 mL.

In some embodiments of each or any of the above or below mentioned embodiments, the first volume of the dermal filler is about 1.5 mL or less, the second volume of the dermal filler is about 2.0 mL or less, and the third volume of the dermal filler is about 1.5 mL or less. In some embodiments, the fourth volume of the dermal filler is about 1 mL or less.

In another aspect of the disclosure, methods of increasing chin protrusion in a subject in need thereof are provided. The methods may generally comprise injecting a first volume of a composition comprising a dermal filler into a pogonion, injecting a second volume of the dermal filler into a mentum, and injecting a third volume of the dermal filler into a left pre-jowl sulcus and a right pre-jowl sulcus, wherein the chin protrusion of the subject is increased after injection of the dermal filler into the pogonion, mentum, left pre jowl sulcus, and right pre-jowl sulcus by at least 2 mm.

In some embodiments of each or any of the above or below mentioned embodiments, the increase in the chin protrusion of the subject is maintained for at least about 1 month, about 2 months, about 3 months, about 4 months, about 5 months, about 6 months, about 7 months, about 8 months, about 9 months, about 10 months, about 11 months, about 12 months, about 13 months, about 14 months, about 15 months, about 16 months, about 17 months, about 18 months, or more.

In some embodiments of each or any of the above or below mentioned embodiments, the chin protrusion is increased by at least about 0.5 mm, about 0.6 mm, about 0.7 mm, about 0.8 mm, about 0.9 mm, about 1 mm, about 1.1 mm, about 1.2 mm, about 1.3 mm, about 1.4 mm, about 1.5 mm, about 1.6 mm, about 1.7 mm. about 1.8 mm, about 1.9 mm, about 2 mm, about 2.1 mm, about 2.2 mm, about 2.3 mm, about 2.4 mm, about 2.5 mm, about 2.6 mm, about 2.7 mm, about 2.8 mm, about 2.9 mm, about 3 mm, about 3.1 mm, about 3.2 mm, about 3.3 mm, about 3.4 mm, about 3.5 mm, about 3.6 mm, about 3.7 mm, about 3.8 mm, about 3.9 mm, about 4 mm, about 4.1 mm, about 4.2 mm, about 4.3 mm, about 4.4 mm, about 4.5 mm, about 4.6 mm, about 4.7 mm, about 4.8 mm, about 4.9 mm, about 5 mm, about 5.1 mm, about 5.2 mm, about 5.3 mm, about 5.4 mm, about 5.5 mm, about 5.6 mm, about 5.7 mm, about 5.8 mm, about 5.9 mm, about 6.0 mm, or more.

In another aspect of the disclosure, methods of injecting a composition comprising a dermal filler are provided. The methods may generally comprise injecting a first volume of about 1.5 mL or less of a dermal filler into a pogonion, injecting a second volume of about 2.0 mL or less of the dermal filler into a mentum, and injecting a third volume of about 1.5 mL or less of the dermal filler into a left pre-jowl sulcus and a right pre-jowl sulcus. In some embodiments of each or any of the above or below mentioned embodiments, the methods may further comprise injecting a fourth volume of about 1 mL or less of the dermal filler into a sublabial crease.

In another aspect of the disclosure, methods of treating chin retrusion in a subject by increasing a glabella-subnasale-pogonion (G-Sn-Pog) facial angle of the subject are provided. The methods may generally comprise injecting a first volume of a composition comprising a dermal filler into a pogonion, injecting a second volume of the dermal filler into a mentum, and injecting a third volume of the dermal filler into a left pre-jowl sulcus and a right pre-jowl sulcus, wherein the G-Sn-Pog facial angle of the subject is increased after injection of the dermal filler into the pogonion, mentum, left pre jowl sulcus, and right pre-jowl sulcus by at least about 1°.

In some embodiments of each or any of the above or below mentioned embodiments, the G-Sn-Pog facial angle of the subject is increased by at least about 2°. In other embodiments of each or any of the above or below mentioned embodiments, an increase in the G-Sn-Pog facial angle of the subject of at least about 1° is maintained for at least about 18 months.

In some embodiments of each or any of the above or below mentioned embodiments, the injection of the dermal filler into the pogonion, mentum, left pre-jowl sulcus, right pre jowl sulcus, and sublabial crease occurs at a first treatment session. In other embodiments of each or any of the above or below mentioned embodiments, the method further comprises a second treatment session about 18 months to about 24 months after the first treatment session. In some embodiments of each or any of the above or below mentioned embodiments, the second treatment session further increases the G-Sn-Pog facial angle of the subject by at least about 1°.

In some embodiments of each or any of the above or below mentioned embodiments, the subject has an initial G-Sn-Pog facial angle of about 145° to about 165°.

In another aspect of the disclosure, methods of treating chin retrusion in a subject in need thereof are provided. The methods may generally comprise injecting a first volume of a composition comprising a dermal filler into a pogonion, injecting a second volume of the dermal filler into a mentum, and injecting a third volume of the dermal filler into a left pre-jowl sulcus and a right pre jowl sulcus, wherein the chin retrusion of the subject is decreased (e.g., the G-Sn-Pog facial angle is increased) after injection of the dermal filler into the pogonion, mentum, left pre jowl sulcus, and right pre jowl sulcus by at least about 2 mm.

In some embodiments of each or any of the above or below mentioned embodiments, the decrease in chin retrusion of the subject is maintained for at least about 1 month, about 2 months, about 3 months, about 4 months, about 5 months, about 6 months, about 7 months, about 8 months, about 9 months, about 10 months, about 11 months, about 12 months, about 13 months, about 14 months, about 15 months, about 16 months, about 17 months, about 18 months, or more.

In some embodiments of each or any of the above or below mentioned embodiments, the chin retrusion is decreased by at least about 0.5 mm, about 0.6 mm, about 0.7 mm, about 0.8 mm, about 0.9 mm, about 1 mm, about 1.1 mm, about 1.2 mm, about 1.3 mm, about 1.4 mm, about 1.5 mm, about 1.6 mm, about 1.7 mm. about 1.8 mm, about 1.9 mm, about 2 mm, about 2.1 mm, about 2.2 mm, about 2.3 mm, about 2.4 mm, about 2.5 mm, about 2.6 mm, about 2.7 mm, about 2.8 mm, about 2.9 mm, about 3 mm, about 3.1 mm, about 3.2 mm, about 3.3 mm, about 3.4 mm, about 3.5 mm, about 3.6 mm, about 3.7 mm, about 3.8 mm, about 3.9 mm, about 4 mm, about 4.1 mm, about 4.2 mm, about 4.3 mm, about 4.4 mm, about 4.5 mm, about 4.6 mm, about 4.7 mm, about 4.8 mm, about 4.9 mm, about 5 mm, about 5.1 mm, about 5.2 mm, about 5.3 mm, about 5.4 mm, about 5.5 mm, about 5.6 mm, about 5.7 mm, about 5.8 mm, about 5.9 mm, about 6.0 mm, or more.

In another aspect of the disclosure, methods of increasing a G-Sn-Pog facial angle in a subject in need thereof are provided. The methods may generally comprise injecting a first volume of a composition comprising a dermal filler into a pogonion, injecting a second volume of the dermal filler into a mentum, and injecting a third volume of the dermal filler into a left pre jowl sulcus and a right pre jowl sulcus, wherein the G-Sn-Pog facial angle of the subject is increased after injection of the dermal filler into the pogonion, mentum, left pre-jowl sulcus, and right pre-jowl sulcus by at least about 1°, wherein the increase in the G-Sn-Pog facial angle of the subject is maintained for at least about 18 months.

Each and every feature described herein, and each and every combination of two or more of such features, is included within the scope of the present disclosure provided that the features included in such a combination are not mutually inconsistent.

### BRIEF DESCRIPTION OF THE DRAWINGS

FIG. 1 shows a facial profile and landmarks for calculating G-Sn-Pog angle of a patient.

FIG. 2 shows the Burstone Angle of an average chin.

FIG. 3 shows possible areas for treatment of the face, including the sublabial crease, pogonion, mentum, and pre jowl sulcus with the compositions disclosed herein.

FIG. 4 shows the change from baseline in the G-Sn-Pog facial angle of subjects after treatment with the compositions disclosed herein.

FIG. 5 shows a G-Sn-Pog facial angle increase of 1.5° in a subject after treatment with the compositions disclosed herein, including injection of approximately 1 mL of the composition into the pogonion.

FIG. 6 shows a G-Sn-Pog facial angle increase of 5° in a subject after treatment with the compositions disclosed herein, including injection of approximately 2 mL of dermal filler into the pogonion.

FIG. 7 shows the change from baseline in chin protrusion of subjects after treatment with the compositions disclosed herein.

FIG. 8 shows the change from baseline in the G-Sn-Pog facial angle of subjects after treatment with the compositions disclosed herein, including the change in the G-Sn-Pog facial angle 1 month after retreatment.

FIG. 9 shows representative images of a subject treated with the compositions disclosed herein at baseline, at 3 months after treatment, and at 18 months after treatment.

FIG. 10 shows the change from baseline in the facial volume of subjects after treatment with the compositions disclosed herein.

FIGS. 11A and 11B show investigator and subject GAIS responder rates after treatment with the compositions disclosed herein.

FIGS. 12A and 12B show FACE-Q satisfaction with chin responder rates after treatment with the compositions disclosed herein.

FIG. 13 shows FACE-Q satisfaction with lower face and jaw responder rates after treatment with the compositions disclosed herein.

FIG. 14 shows FACE-Q psychological well-being responder rates after treatment with the compositions disclosed herein.

FIG. 15 shows the incidence, severity, and duration of injection site reactions following initial and repeat treatment with the compositions disclosed herein.

### DETAILED DESCRIPTION

Certain terms as used in the specification are intended to refer to the following definitions, as detailed below. Where the definition of terms departs from the commonly used meaning of the term, applicant intends to utilize the definitions provided below, unless specifically indicated.

The term “about” in the context of numerical values will be readily understood by a person skilled in the art, and preferably means that specific values may be modified by +/−10%. As regards endpoints of ranges, the modifier “about” preferably means that the lower endpoint may be reduced by 10% and the upper endpoint increased by 10%. It is also contemplated that each numerical value or range disclosed in this application can be absolute, i.e. that the modifier “about” can be deleted.

All numbers herein expressing “molecular weight” of HA are to be understood as indicating the weight average molecular weight (Mw) in Daltons.

The molecular weight of HA is calculated from an intrinsic viscosity measurement using the following Mark Houwink relation:

Intrinsic Viscosity (L/g)=9.78×10−5×Mw0.690

The intrinsic viscosity is measured according to the procedure defined European Pharmacopoeia (HA monograph No 1472, Janurary 2009).

Unless stated otherwise, the molecular weight refers to the weight average molecular weight (Mw). The HA used to make the present compositions may comprise a mixture of high molecular weight HA, low molecular weight HA, and/or medium molecular weight HA, wherein the high molecular weight HA has a molecular weight greater than about 2,000,000 Da (or an intrinsic viscosity greater than 2.2 L/g) and wherein the low molecular weight HA has a molecular weight of less than about 1,000,000 Da (or an intrinsic viscosity lower than 1.4 L/g). For example, the high molecular weight HA in the present compositions may have an average molecular weight in the range about 2 MDa to about 4.0 MDa, for example, about 3.0 MDa (2.9 L/g). In another example, the high molecular weight HA may have an average molecular weight of between about 2.4 MDa to about 3.6 MDa, for example, about 3.0 MDa. The high molecular weight HA may have an intrinsic viscosity greater than about 2.2 L/g, for example, between about 2.5 L/g to about 3.3 L/g.

Low molecular weight HA can have a molecular weight of between about 200,000 Da (0.2 MDa) to less than 1.0 MDa, for example, between about 300,000 Da (0.3 MDa) to about 750,000 Da (1.1 L/g), up to but not exceeding 0.99 MDa (1.4 L/g). The low molecular weight HA may have an intrinsic viscosity of less than about 1.40 L/g, for example, between about 0.6 L/g and about 1.2 L/g.

Preferably, there is no overlap between the molecular weight distribution of the low and high molecular weight HA materials.

Preferably, the mixture of the low molecular weight HA and high molecular weight HA has a bimodal molecular weight distribution. The mixture may also have a multi-modal distribution.

In one aspect of the disclosure, the compositions comprise HA having a high molecular weight component and a low molecular weight component, and the high molecular weight component has a weight average molecular weight at least twice the weight average molecular weight of the low molecular weight component.

“Degree of crosslinking” as used herein refers to the intermolecular junctions joining the individual HA polymer molecules, or monomer chains, into a permanent structure, or as disclosed herein the soft tissue filler composition. Moreover, degree of crosslinking for purposes of the present disclosure is further defined as the percent weight ratio of the crosslinking agent to HA-monomeric units within the crosslinked portion of the HA based composition. It is measured by the weight ratio of crosslinker to HA monomers.

“Uncrosslinked HA” as used herein refers to individual HA polymer molecules that are not crosslinked. Uncrosslinked HA generally remains water soluble. An uncrosslinked HA fraction may optionally also be included in the compositions, for example, to act as a lubricant and facilitate injection into the facial tissues. Such a composition may comprise an uncrosslinked HA fraction where the added uncrosslinked HA is present at a concentration between about 0.1 mg/g and about 3 mg/g. Preferably, the uncrosslinked HA may be present at a concentration between about 0.2 mg/g and about 1.5 mg/g.

In other embodiments, no uncrosslinked HA is present in the gels, or at least no uncrosslinked HA is added to the gels to act as a lubricant.

The compositions described herein display a high level of elasticity, expressed as a value of elastic modulus (G′) measured by oscillation rheology with a strain of 0.8%, using a cone-plate system and measured over a range of frequencies. In some embodiments, the elastic modulus of the compositions measured at 5 Hz frequency are from about 500 Pa to about 900 Pa. This is considered as high elasticity in the context of HA-based dermal fillers and contributes to the lifting effect by making the implant more resistant to shear deformation.

Cohesivity refers to the capacity of the gel to stay attached to itself, for example, meaning the resistance to cutting and the ability to elongate or compress the gel without it separating into pieces. The cohesivity of the gels according to the present disclosure can be quantified as follows (cf. Derek Jones “Injectable Filers: Principles and Practice”, Wiley, 2011, Chapter 3). A small sample of the gel (e.g. 1 mL) is placed onto the plane surface of a rheometer. The sample is placed such that it forms a little heap. A moveable upper plate is placed onto the sample so that the sample is fully covered, e.g. when looking at the plate in a direction perpendicular to the surface of the rheometer, the sample cannot be seen. In order to ensure this, one must choose a plate size that is larger than the sample size. Ideally, the center of the plate is placed over the sample. Typically, for 1 mL of gel material, a 25 mm diameter upper plate is used.

In the next step of the measurement, one then adjusts the gap between the moveable plate and the surface to 2.5 mm. While slowly and steadily moving the plate from this initial position towards a gap width of 0.9 mm within 2 min one records the force (Fn) exerted by the sample in normal direction on the plate.

Once a gap width of 0.9 mm is reached, the system is allowed to relax for 12 minutes. During this time, the measurement is continued. Five measurements are done. To normalize the forces measured, all 5 initial Fn values measured when the test starts are averaged (arithmetic mean) and this resulting average is subtracted from all other data points. The maximum force at the end on the compressive part of this test (when reaching the minimal 0.9 mm gap width between the upper plate and the plane) is called the compression force and is the characteristic value for determining the cohesivity of the gel.

Specifically, a force of 20 gmf (0.1962 N) or more indicates a cohesive material in the sense of the present disclosure. Gels with lower compression force values are generally not considered cohesive in the context of the present disclosure. The accuracy of this measurement is in the order of ±5 gmf. In the context of this disclosure, the injectable formulation has a high cohesivity of at least about 60 gmf, for example about 60 to about 200 gmf. For example, in a preferred embodiment, cohesivity is between about 60 and about 100 gmf, which will give to the implant a high resistance to pressure and normal forces in the soft tissues of the face.

In the context of a dermal filler, the cohesivity as defined above will contribute to the lift capacity (clinically called the volumizing/bulking effect) provided by the gel clinically, along with its elastic modulus G′. While cohesive gels can show a good volumizing effect, non-cohesive or weakly cohesive materials with a similar elastic modulus exhibits lower lift capacity due to the non-cohesive gel material spreading more than a more cohesive material when submitted to vertical compression. In the context of this disclosure, the compositions exhibit both high levels of elastic modulus and high levels of cohesivity, to maximize the lifting effect upon implantation.

In certain advantageous, exemplary embodiments, the present implants or fillers generally comprise a cohesive, sterile composition which is implantable subdermally or supraperiostially into the chin area, nose or jawline of the patient in need thereof, for example a patient desiring an improved facial profile or stronger chin. The composition generally comprises a crosslinked hyaluronic acid (HA) crosslinked with 1,4-butanediol diglycidyl ether (BDDE); and the HA concentration of the composition is greater than 20 mg/g. For example, in some embodiments, the HA concentration is about 22.5 mg/g, or about 25 mg/g, or about 27.5 mg/g. The HA used for crosslinking may be made with a mixture of low molecular weight hyaluronic acid and high molecular weight hyaluronic acid. In some embodiments, the compositions have an elastic modulus between about 500 Pa and about 900 Pa at 5 Hz, and a cohesivity above about 60 gmf. Advantageously, in some embodiments, the compositions exhibit an extrusion force between about 4N and about 15N, for example, between about 8N and about 10 N, at 13 mm/min using a 1 mL COC syringe and a 27 G×13 mm needle.

In one aspect of the disclosure, injectable HA-based implants having an improved lift capacity, relative to commercial HA-based dermal fillers, are provided. The present implants are, in some instances in the present disclosure, referred alternatively as dermal fillers and subdermal fillers. The implants and fillers of the present disclosure are based on hyaluronic acids (HA) and pharmaceutically acceptable salts of HA, for example, sodium hyaluronate (NaHA). Methods of making these compositions, and methods of use of these compositions, are also provided.

As used herein, hyaluronic acid (HA) can refer to any of its hyaluronate salts, and includes, but is not limited to, sodium hyaluronate (NaHA), potassium hyaluronate, magnesium hyaluronate, calcium hyaluronate, and combinations thereof. Both HA and pharmaceutically acceptable salts thereof can be used in this disclosure.

In addition, in embodiments with anesthetics, the concentration of one or more anesthetics is in an amount effective to mitigate pain experienced upon injection of the composition. The at least one local anesthetic can be selected from the group of ambucaine, amolanone, amylocaine, benoxinate, benzocaine, betoxycaine, biphenamine, bupivacaine, butacaine, butamben, butanilicaine, butethamine, butoxycaine, carticaine, chloroprocaine, cocaethylene, cocaine, cyclomethycaine, dibucaine, dimethisoquin, dimethocaine, diperodon, dicyclomine, ecgonidine, ecgonine, ethyl chloride, etidocaine, beta-eucaine, euprocin, fenalcomine, formocaine, hexylcaine, hydroxytetracaine, isobutyl p-aminobenzoate, leucinocaine mesylate, levoxadrol, lidocaine, mepivacaine, meprylcaine, metabutoxycaine, methyl chloride, myrtecaine, naepaine, octocaine, orthocaine, oxethazaine, parethoxycaine, phenacaine, phenol, piperocaine, piridocaine, polidocanol, pramoxine, prilocaine, procaine, propanocaine, proparacaine, propipocaine, propoxycaine, pseudococaine, pyrrocaine, ropivacaine, salicyl alcohol, tetracaine, tolycaine, trimecaine, zolamine, and salts thereof. In one embodiment, the at least one anesthetic agent is lidocaine, such as in the form of lidocaine HCl. The compositions described herein may have a lidocaine concentration of between about 0.1% and about 5% by weight of the composition, for example, about 0.2% to about 1.0% by weight of the composition. In one embodiment, the composition has a lidocaine concentration of about 0.3% by weight (w/w %) of the composition. The concentration of lidocaine in the compositions described herein can be therapeutically effective meaning the concentration is adequate to provide a therapeutic benefit without inflicting harm to the patient.

The present compositions may be manufactured by the steps of providing purified HA material for example, in the form of NaHA fibers; the HA material having a desired molecular weight, for example, a mixture of low molecular weight and high molecular weight HA at a desired ratio, hydrating the HA material; and crosslinking the hydrated HA material with a suitable crosslinking agent at the desired ratio to form a crosslinked HA-based gel. The gel may then be neutralized and swollen. If desired, a solution containing lidocaine, preferably an acidic salt of lidocaine chlorohydrate, may be added to form a HA/lidocaine gel. The gel may be homogenized, for example, by beating or mixing with a shear force. The homogenized composition may then be packaged in syringes. The syringes are then sterilized by autoclaving at an effective temperature and pressure. For example, the compositions are sterilized by autoclaving, for example, being exposed to temperatures of at least about 120° C. to about 130° C. and/or pressures of at least about 12 pounds per square inch (PSI) to about 20 PSI for a period of at least about 1 minute to about 15 minutes. The sterilized syringes are packaged along with a fine gauge needle for use by a physician.

More specifically, the initial raw HA material may comprise fibers or powder of NaHA, for example, bacterial-sourced NaHA fibers. Alternatively, the HA material may be animal derived, for example, from rooster combs. It is contemplated that the HA material may be a combination of raw materials including HA and at least one other polysaccharide, for example, another glycosaminoglycan (GAG).

In one method of manufacturing the compositions, pure, dry NaHA fibers are hydrated in an alkaline solution to produce an uncrosslinked NaHA gel. Any suitable alkaline solution may be used to hydrate the NaHA in this step, for example, but not limited to aqueous solutions containing sodium hydroxide (NaOH), potassium hydroxide (KOH), sodium bicarbonate (NaHCO₃), lithium hydroxide (LiOH), and the like. The resulting alkaline gel will have a pH above 7.5. The pH of the resulting alkaline gel can have a pH greater than 9, or a pH greater than 10, or a pH greater than 12, or a pH greater than 13.

The next step in the manufacturing process may include the step of crosslinking the hydrated, alkaline NaHA gel with a suitable crosslinking agent. The crosslinking agent may be any agent known to be suitable for crosslinking polysaccharides and their derivatives via their hydroxyl groups. One particular suitable crosslinking agent is 1,4-butanediol diglycidyl ether (BDDE).

In another embodiment of each or any of the above or below mentioned embodiments, the crosslinking of the HA is accomplished during hydration of the HA fibers, by hydrating the combined high and low molecular weight fibers in an alkaline solution containing a crosslinking agent, for example, BDDE.

The degree of crosslinking in the HA component of the present compositions is at least about 4% and is up to about 12% BDDE/HA, w/w, for example, about 10%, for example, about 8%, for example, about 6%, for example, about 4%. In a specific embodiment, the degree of crosslinking is about 6.5%. In some embodiments the HA has a degree of crosslinking of about 6.5%. In other embodiments, the HA has a degree of crosslinking of about 7.5%, or about 8.5%, or about 9.5%, or about 10.5%.

The hydrated crosslinked, HA gels may be swollen to obtain the desired HA concentration. This step can be accomplished by neutralizing the crosslinked, hydrated HA gel, for example by adding an aqueous solution containing of an acid, such as HCl. The gels are then swelled in a phosphate buffered saline (PBS) solution for a sufficient time and at a low temperature.

The gels may now be purified by conventional means such as, dialysis against a phosphate buffer, or alcohol precipitation, to recover the crosslinked material, to stabilize the pH of the material and to remove any un-reacted crosslinking agent. Additional water or a slightly alkaline aqueous solution can be added to bring the concentration of the HA in the composition to a desired concentration. In some embodiments, the HA concentration of the compositions is adjusted to above 20 mg/g, for example, to about 25 mg/g. In other embodiments, the HA concentration is adjusted to yield an HA concentration of about 21 mg/g, about 22 mg/g, about 23 mg/g, about 24 mg/g, about 26 mg/g, about 27 mg/g, about 28 mg/g, about 29 mg/g, or about 30 mg/g.

In embodiments in which an anesthetic agent is to be included in the final composition, such as lidocaine, the pH of the purified crosslinked HA gels may be adjusted to cause the gel to become slightly alkaline such that the gels have a pH of greater than about 7.2, for example, about 7.5 to about 8.0. This step may be accomplished by any suitable means, for example, by adding a suitable amount of dilute NaOH, KOH, NaHCO3 or LiOH, to the gels or any other alkaline molecule, solution and/or buffering composition.

An effective amount of the anesthetic, for example, lidocaine, such as lidocaine HCl, is then added to the purified crosslinked NaHA gels. For example, in some embodiments, the lidocaine HCl is provided in a powder form which is solubilized using water for injection (WFI). The gels are kept neutral with a buffer or by adjustment with diluted NaOH in order that the final HA/lidocaine composition will have a desired, substantially neutral pH. The final compositions including lidocaine may have a lidocaine concentration of between at least about 0.1% and about 5%, for example, about 2% by weight of the composition, or in another example about 0.3%.

After the addition of the lidocaine HCl, or alternatively, during the addition of the lidocaine HCl, the HA/lidocaine gels, or compositions, are homogenized to create highly homogenous HA/lidocaine gels having a desired consistency and stability. Preferably, the homogenization step comprises mixing, stirring, or beating the gels with a controlled shearing force obtaining substantially homogenous mixtures.

After homogenizing the HA composition, an amount of uncrosslinked HA solution or gel may be added to the composition to increase lubricity.

In some embodiments of each or any of the above or below mentioned embodiments, no solution of uncrosslinked HA is added to the composition after homogenization.

The compositions may then be introduced into syringes and sterilized. Syringes useful according to the present description include any syringe known in the art capable of delivering viscous dermal filler compositions. The syringes generally have an internal volume of about 0.4 mL to about 3 mL, more preferably between about 0.5 mL and about 1.5 mL or between about 0.8 mL and about 2.5 mL. This internal volume is associated with an internal diameter of the syringe which plays a key role in the extrusion force needed to inject high viscosity dermal filler compositions. The internal diameters are generally about 4 mm to about 9 mm, more preferably from about 4.5 mm to about 6.5 mm or from about 4.5 mm to about 8.8 mm. Further, the extrusion force needed to deliver the HA compositions from the syringe is dependent on the needle gauge. The gauges of needles used generally include gauges between about 18 G and about 40 G, more preferably about 25 G to about 33 G, or from about 25 G to about 30 G. For example, in some embodiments, the compositions are packaged in a 1 mL syringe and injected using a 27 G needle.

One preferable method of sterilization of the filled syringes is by autoclave. Autoclaving can be accomplished by applying a mixture of heat, pressure and moisture to a sample in need of sterilization. Many different sterilization temperatures, pressures and cycle times can be used for this step. For example, the filled syringes may be sterilized at a temperature of at least about 120° C. to about 130° C. or greater. Moisture may or may not be utilized. The pressure applied is in some embodiments depending on the temperature used in the sterilization process. The sterilization cycle may be at least about 1 minute to about 20 minutes or more.

Another method of sterilization incorporates the use of a gaseous species which is known to kill or eliminate transmissible agents. Preferably, ethylene oxide is used as the sterilization gas and is known in the art to be useful in sterilizing medical devices and products.

A further method of sterilization incorporates the use of an irradiation source which is known in the art to kill or eliminate transmissible agents. A beam of irradiation is targeted at the syringe containing the HA composition, and the wavelength of energy kills or eliminates the unwanted transmissible agents. Preferable energy useful include, but is not limited to ultraviolet (UV) light, gamma irradiation, visible light, microwaves, or any other wavelength or band of wavelengths which kills or eliminates the unwanted transmissible agents, preferably without substantially altering of degrading the HA composition.

Preferably, the present compositions also remain stable when stored for long periods of time. For example, many of the present compositions have a shelf life of about 6 months, about 12 months, about 18 months, or about 24 months or greater, when stored at a temperature between about 2 to 25 degrees C. In a specific embodiment, the compositions are stable at a temperature of between 2 to 25 degrees C. for a period of at least 18 months. In another specific embodiment, the compositions are stable at a temperature or between 2 to 25 degrees C. for a period of at least 24 months.

In one aspect of the disclosure, methods of increasing a glabella-subnasale-pogonion (G-Sn-Pog) facial angle in a subject in need thereof are provided. The methods may comprise injecting a first volume of a composition comprising a dermal filler into a pogonion, injecting a second volume of the dermal filler into a mentum, and/or injecting a third volume of the dermal filler into a left pre-jowl sulcus and/or a right pre-jowl sulcus, wherein the G-Sn-Pog facial angle of the subject is increased after injection of the dermal filler into the pogonion, mentum, left pre-jowl sulcus, and/or right pre-jowl sulcus by at least about 1° or more.

In some embodiments of each or any of the above or below mentioned embodiments, the increase in the G-Sn-Pog facial angle of the subject is maintained for at least about 1 month, about 2 months, about 3 months, about 4 months, about 5 months, about 6 months, about 7 months, about 8 months, about 9 months, about 10 months, about 11 months, about 12 months, about 13 months, about 14 months, about 15 months, about 16 months, about 17 months, about 18 months, or more.

In some embodiments of each or any of the above or below mentioned embodiments, the increase in the G-Sn-Pog facial angle of the subject is increased by at least about 0.5°, about 0.6°, about 0.7°, about 0.8°, about 0.9°, about 1°, about 1.1°, about 1.2°, about 1.3°, about 1.4°, about 1.5°, about 1.6°, about 1.7°, about 1.8°, about 1.9°, about 2°, about 2.1°, about 2.2°, about 2.3°, about 2.4°, about 2.5°, about 2.6°, about 2.7°, about 2.8°, about 2.9°, about 3°, about 3.1°, about 3.2°, about 3.3°, about 3.4°, about 3.5°, about 3.6°, about 3.7°, about 3.8°, about 3.9°, about 4°, about 4.1°, about 4.2°, about 4.3°, about 4.4°, about 4.5°, about 4.6°, about 4.7°, about 4.8°, about 4.9°, about 5°, about 5.1°, about 5.2°, about 5.3°, about 5.4°, about 5.5°, about 5.6°, about 5.7°, about 5.8°, about 5.9°, about 6.0°, or more.

In some embodiments of each or any of the above or below mentioned embodiments, the method further comprises injecting a fourth volume of the dermal filler into a sublabial crease.

In another aspect of the disclosure, methods of increasing chin protrusion in a subject in need thereof are disclosed. The methods may comprise injecting a first volume of a composition comprising a dermal filler into a pogonion, injecting a second volume of the dermal filler into a mentum, and/or injecting a third volume of the dermal filler into a left pre-jowl sulcus and/or a right pre-jowl sulcus, wherein the chin protrusion of the subject is increased after injection of the dermal filler into the pogonion, mentum, left-pre jowl sulcus, and/or right pre jowl sulcus by at least about 2 mm or more.

In some embodiments of each or any of the above or below mentioned embodiments, the increase in the chin protrusion of the subject is maintained for at least about 1 month, about 2 months, about 3 months, about 4 months, about 5 months, about 6 months, about 7 months, about 8 months, about 9 months, about 10 months, about 11 months, about 12 months, about 13 months, about 14 months, about 15 months, about 16 months, about 17 months, about 18 months, or more.

In some embodiments of each or any of the above or below mentioned embodiments, the chin protrusion is increased by at least about 0.5 mm, about 0.6 mm, about 0.7 mm, about 0.8 mm, about 0.9 mm, about 1 mm, about 1.1 mm, about 1.2 mm, about 1.3 mm, about 1.4 mm, about 1.5 mm, about 1.6 mm, about 1.7 mm. about 1.8 mm, about 1.9 mm, about 2 mm, about 2.1 mm, about 2.2 mm, about 2.3 mm, about 2.4 mm, about 2.5 mm, about 2.6 mm, about 2.7 mm, about 2.8 mm, about 2.9 mm, about 3 mm, about 3.1 mm, about 3.2 mm, about 3.3 mm, about 3.4 mm, about 3.5 mm, about 3.6 mm, about 3.7 mm, about 3.8 mm, about 3.9 mm, about 4 mm, about 4.1 mm, about 4.2 mm, about 4.3 mm, about 4.4 mm, about 4.5 mm, about 4.6 mm, about 4.7 mm, about 4.8 mm, about 4.9 mm, about 5 mm, about 5.1 mm, about 5.2 mm, about 5.3 mm, about 5.4 mm, about 5.5 mm, about 5.6 mm, about 5.7 mm, about 5.8 mm, about 5.9 mm, about 6.0 mm, or more.

In yet another aspect of the disclosure, methods of injecting a composition comprising a dermal filler are provided. The methods may comprise injecting a first volume of about 1.5 mL or less of a dermal filler into a pogonion, injecting a second volume of about 2.0 mL or less of the dermal filler into a mentum, and/or injecting a third volume of about 1.5 mL or less of the dermal filler into a left pre-jowl sulcus and/or a right pre-jowl sulcus. In some embodiments of each or any of the above or below mentioned embodiments, the methods may further comprise injecting a fourth volume of about 1 mL or less of the dermal filler into a sublabial crease.

In another aspect of the disclosure, methods of treating chin retrusion in a subject by increasing a glabella-subnasale-pogonion (G-Sn-Pog) facial angle of the subject are provided. The methods may generally comprise injecting a first volume of a composition comprising a dermal filler into a pogonion, injecting a second volume of the dermal filler into a mentum, and injecting a third volume of the dermal filler into a left pre-jowl sulcus and a right pre-jowl sulcus, wherein the G-Sn-Pog facial angle of the subject is increased after injection of the dermal filler into the pogonion, mentum, left pre jowl sulcus, and right pre-jowl sulcus by at least about 1°.

In some embodiments of each or any of the above or below mentioned embodiments, the G-Sn-Pog facial angle of the subject is increased by at least about 2°. In other embodiments of each or any of the above or below mentioned embodiments, an increase in the G-Sn-Pog facial angle of the subject of at least about 1° is maintained for at least about 18 months.

In some embodiments of each or any of the above or below mentioned embodiments, the injection of the dermal filler into the pogonion, mentum, left pre-jowl sulcus, right pre jowl sulcus, and sublabial crease occurs at a first treatment session. In other embodiments of each or any of the above or below mentioned embodiments, the method further comprises a second treatment session about 18 months to about 24 months after the first treatment session. In some embodiments of each or any of the above or below mentioned embodiments, the second treatment session further increases the G-Sn-Pog facial angle of the subject by at least about 1°.

In some embodiments of each or any of the above or below mentioned embodiments, the subject has an initial G-Sn-Pog facial angle of about 145° to about 165°.

In another aspect of the disclosure, methods of treating chin retrusion in a subject in need thereof are provided. The methods may generally comprise injecting a first volume of a composition comprising a dermal filler into a pogonion, injecting a second volume of the dermal filler into a mentum, and injecting a third volume of the dermal filler into a left pre-jowl sulcus and a right pre jowl sulcus, wherein the chin retrusion of the subject is decreased (e.g., the G-Sn-Pog facial angle is increased) after injection of the dermal filler into the pogonion, mentum, left pre jowl sulcus, and right pre jowl sulcus by at least about 2 mm.

In some embodiments of each or any of the above or below mentioned embodiments, the decrease in chin retrusion of the subject is maintained for at least about 1 month, about 2 months, about 3 months, about 4 months, about 5 months, about 6 months, about 7 months, about 8 months, about 9 months, about 10 months, about 11 months, about 12 months, about 13 months, about 14 months, about 15 months, about 16 months, about 17 months, about 18 months, or more.

In some embodiments of each or any of the above or below mentioned embodiments, the chin retrusion is decreased by at least about 0.5 mm, about 0.6 mm, about 0.7 mm, about 0.8 mm, about 0.9 mm, about 1 mm, about 1.1 mm, about 1.2 mm, about 1.3 mm, about 1.4 mm, about 1.5 mm, about 1.6 mm, about 1.7 mm. about 1.8 mm, about 1.9 mm, about 2 mm, about 2.1 mm, about 2.2 mm, about 2.3 mm, about 2.4 mm, about 2.5 mm, about 2.6 mm, about 2.7 mm, about 2.8 mm, about 2.9 mm, about 3 mm, about 3.1 mm, about 3.2 mm, about 3.3 mm, about 3.4 mm, about 3.5 mm, about 3.6 mm, about 3.7 mm, about 3.8 mm, about 3.9 mm, about 4 mm, about 4.1 mm, about 4.2 mm, about 4.3 mm, about 4.4 mm, about 4.5 mm, about 4.6 mm, about 4.7 mm, about 4.8 mm, about 4.9 mm, about 5 mm, about 5.1 mm, about 5.2 mm, about 5.3 mm, about 5.4 mm, about 5.5 mm, about 5.6 mm, about 5.7 mm, about 5.8 mm, about 5.9 mm, about 6.0 mm, or more.

In another aspect of the disclosure, methods of increasing a G-Sn-Pog facial angle in a subject in need thereof are provided. The methods may generally comprise injecting a first volume of a composition comprising a dermal filler into a pogonion, injecting a second volume of the dermal filler into a mentum, and injecting a third volume of the dermal filler into a left pre jowl sulcus and a right pre jowl sulcus, wherein the G-Sn-Pog facial angle of the subject is increased after injection of the dermal filler into the pogonion, mentum, left pre-jowl sulcus, and right pre-jowl sulcus by at least about 1°, wherein the increase in the G-Sn-Pog facial angle of the subject is maintained for at least about 18 months.

The technique for injection of the present compositions may vary with regard to the angle and orientation of the bevel, and the quantity administered. In general, the present compositions are injected subcutaneously and/or supraperiosteally to increase chin projection, limiting treatment to the pogonion, the mentum (inferior aspect of the chin), pre-jowl sulci (left and right), and sublabial (mental) crease to achieve optimal correction and aesthetic chin contour. The appropriate injection volume will be determined by the investigator but is generally not to exceed a maximum total volume of about 4.0 mL for initial and top-up treatments combined. Up to about 4.0 mL total is allowed for repeat treatment. No more than about 2.0 mL is permitted to be injected into a single treatment area at any treatment session, where treatment areas are defined as the pogonion, the mentum, the pre jowl sulci (left and right), and the sublabial (mental) crease.

In some embodiments of each or any of the above or below mentioned embodiments, the total volume of the dermal filler injected into the pogonion, mentum, left pre-jowl sulcus, right pre-jowl sulcus, and/or sublabial crease is no greater than about 3.0 mL, about 4.0 mL, or about 5.0 mL.

In some embodiments of each or any of the above or below mentioned embodiments, each injection volume is no greater than about 2.0 mL.

In some embodiments of each or any of the above or below mentioned embodiments, the first volume of the dermal filler is about 1.5 mL or less, the second volume of the dermal filler is about 2.0 mL or less, and the third volume of the dermal filler is about 1.5 mL or less. In some embodiments, the fourth volume of the dermal filler is about 1 mL or less.

Prior to injection of the present compositions, the treatment area has to be thoroughly disinfected to ensure that there is no contamination of the injectable filler with bacteria or a foreign body (e.g., make-up, talc from gloves).

Next, the 27 G ½″/27 G×13 mm needle supplied should be attached to the syringe (according to Directions for Use). Prior to injecting the present compositions, the plunger rod has to be depressed until the product visibly flows out of the needle and wipe any excess on sterile gauze.

The present compositions are injected as follows: Inject the present compositions slowly, and observe the skin for signs of colour change or discolouration. Observe the subject for pain or discomfort. Inject the present compositions in a smooth and measured manner. Insert the needle being mindful of the local vascular anatomy at the injection site. Aspirate to ensure there is no blood backflow to suggest an intravascular location of the tip of the needle.

In some embodiments of each or any of the above or below mentioned embodiments, the pogonion may be injected supraperiosteally using multiple small boluses. In some embodiments, the mentum may be injected supraperiosteally using multiple small boluses. In some embodiments, the pre jowl sulci (e.g., the left and/or right sulcus) may be injected using a deep subcutaneous fanning technique. In some embodiments, the sublabial (mental) crease may be injected using linear, retrograde or anterograde superficial subcutaneous threading.

In some embodiments of each or any of the above or below mentioned embodiments, the injections into the pogonion, mentum, left pre jowl sulcus, and/or right pre-jowl sulcus comprise a serial puncture technique. In some embodiments, the injections into the pogonion, mentum, left pre-jowl sulcus, and/or right pre-jowl sulcus comprise a supraperiosteal injection. In some embodiments, the injection into the sublabial crease comprises a subcutaneous injection.

When treatment is completed, the treated site may be gently massaged to assure that the product is evenly distributed and conforms to the contour of the surrounding tissues. If overcorrection occurs, gently massage the area between your fingers or against an underlying bone to obtain optimal results.

The present compositions are not to be injected into the blood vessels (intravascular). Introduction of hyaluronic acid into the vasculature may occlude the vessels and could cause infarction or embolization. Symptoms of vascular occlusions and embolization include pain that is disproportionate to the procedure or remote to the injection site, immediate blanching that extends beyond the injected area and that may represent vascular tributary distribution, and colour changes that reflect ischemic tissue such as a dusky or reticular appearance.

Injecting the product too superficially or in large volumes over a small area may result in visible and persistent lumps and/or discoloration.

When using a retrograde technique, inject the present compositions applying even pressure on the plunger rod while slowly pulling the needle backward. It is important that the injection be stopped just before the needle passes the subcutaneous/dermal interface to prevent material from leaking out or ending up too superficially in the skin. When using an anterograde technique, be sure the needle is in the subcutaneous tissue before the injection is started.

If the needle is blocked, do not increase the pressure on the plunger rod but stop the injection and replace the needle.

If the treated area is swollen immediately after the injection, an ice pack may be applied to the site for a short period. If subjects report inflammatory reactions which persist for more than 1 week, or any other side effect which develops, the medical practitioner should use an appropriate treatment.

In preferred embodiments, the present compositions comprise a hyaluronic acid gel, preferably in an amount of about 25 mg; and lidocaine hydrochloride, preferably in an amount of about 3 mg, in a phosphate buffer (pH 7.2), preferably in a volume q.s. 1 mL, prefilled in e.g. a 1 mL single-use syringe, wherein the hyaluronic acid gel is crosslinked with BDDE. This prefilled e.g. 1 mL single-use syringe may be contained in a kit (blister pack) along with two single use needles (e.g. 27 G ½″/27 G×13 mm needles). The content of the syringe may be sterilised by moist heat. The single-use needles may be sterilised by radiation.

The present compositions are injectable implants intended for restoration and creation of facial volume, e.g. in the chin and jaw area. The presence of lidocaine is meant to reduce the subject's pain during treatment.

In yet another aspect of the disclosure, methods of treating a subject to increase his or her G-Sn-Pog facial angle are provided. The methods may comprise at a first treatment session injecting at least one volume of a composition comprising a dermal filler into one or more of a pogonion, mentum, left pre-jowl sulcus, right pre-jowl sulcus, and/or sublabial crease; and at a second treatment session about 5 days, about 10 days, about 15 days, about 20 days, about 25 days, or about 30 days after the first treatment session injecting at least one volume of the dermal filler into one or more of the pogonion, mentum, left pre-jowl sulcus, right pre-jowl sulcus, and/or sublabial crease. In some embodiments, the subject's G-Sn-Pog facial angle is increased by at least about 1° or more.

In yet another aspect of the disclosure, methods of treating a subject to increase his or her chin protrusion are provided. The methods may comprise at a first treatment session injecting at least one volume of a composition comprising a dermal filler into one or more of a pogonion, mentum, left pre jowl sulcus, right pre jowl sulcus, and/or sublabial crease; and at a second treatment session about 5 days, about 10 days, about 15 days, about 20 days, or about 30 days after the first treatment session injecting at least one volume of the dermal filler into one or more of the pogonion, mentum, left pre jowl sulcus, right pre jowl sulcus, and/or sublabial crease. In some embodiments, the subject's chin protrusion is increased by at least about 2 mm or more.

### Example 1

### Manufacture of an Injectable Implant in Accordance with Some Embodiments

Predried fibers of sodium hyaluronate (NaHA) (0.9 g) having a molecular weight of about 0.9 MDa is weighed out into a first receptacle.

Predried fibers of NaHA (0.1 g) having a molecular weight of about 3.0 MDa is weighed out into a second receptacle.

The two different grades of NaHA are combined and diluted into a 1% sodium hydroxide solution and mixed for one to two hours at between 20° C. and 50° C. to obtain a substantially homogenous, alkaline HA gel.

In a separate receptacle, the chosen crosslinking agent, 1,4-butanediol diglycidyl ether (BDDE), is diluted into a 1% sodium hydroxide solution to a final concentration of 10% BDDE (wt/wt).

To the alkaline HA gel was added 10% (wt/wt) BDDE (1 g of the previously prepared BDDE solution). The resulting mixture is mechanically homogenized.

The mixture is then maintained at 50° C. for 3 to 4.5 hours.

The resulting crosslinked HA polymer is then immersed in a phosphate buffer (PB) containing hydrochloric acid to stabilize the pH.

The crosslinked HA polymer so obtained is then immersed in baths of phosphate buffer to remove unreacted crosslinking agent and HA, providing the purified hydrogel, wherein the degree of crosslinking is about 6.5%.

Optionally, dry HA material having a high molecular weight is hydrated in 1 liter of phosphate buffer to obtain an uncrosslinked HA gel. This uncrosslinked HA gel can be added to the crosslinked HA composition to represent up to 5% (w/w) of the total HA concentration.

The hydrogel obtained is then homogenized mechanically to ensure the final homogeneity, and packed into syringes which are sterilized in an autoclave.

The gel obtained is an injectable composition that can be administered subdermally or supraperiostally through a fine gauge needle (e.g. 27 Gauge). The composition is useful for restoring, contouring, or creating facial volume, for example, in the chin, jaw area, or nose of a person, as described elsewhere herein.

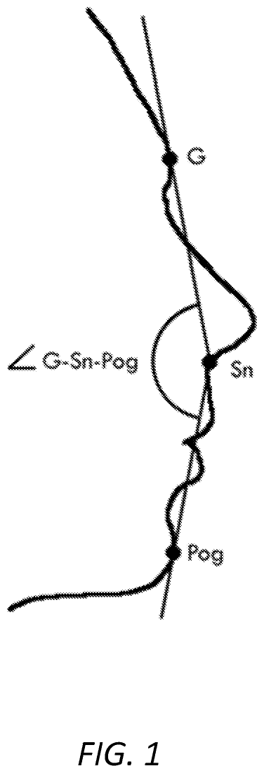

In one aspect of the disclosure, methods are provided for improving a subject's facial profile. For example, in some embodiments, methods are provided for changing a subject's G-Sn-Pog facial angle, for example, for increasing a subject's G-Sn-Pog facial angle. For example, in some embodiments, methods of treatment are provided for correcting chin retrusion in a subject. In some embodiments of the disclosure, the subject treated has an initial pre-treatment G-Sn-Pog facial angle of less than about 165°. After the treatment, the subject has an increased G-Sn-Pog facial angle, that is, a facial angle greater than the initial pre-treatment facial angle. In one embodiment, the subject has a G-Sn-Pog angle of about 169° or greater after the step of administering. The G-Sn-Pog angle may be measured using conventional equipment and calculations, for example, may be based on calculations of facial angle derived from digital images of the subject, for example, using Canfield scientific facial imaging equipment. FIG. 1 shows facial profile and landmarks for calculating G-Sn-Pog angle of a subject, which can be used to diagnose or determine the presence and/or degree of chin retrusion, using known methods.

The methods may generally comprise administering into at least one treatment area of the face of the subject, an effective amount of a composition comprising BDDE-crosslinked hyaluronic acid (HA), the HA having a degree of crosslinking of about 6.5%, or about 10%, and having a HA concentration of greater than 20 mg/g.

In some embodiments, treatment methods are provided, the methods may comprise supraperiosteally administering a composition, such as described herein, into at least one treatment area of the face of a patient, wherein the patient has a G-Sn-Pog facial angle of 145° to 165°. The facial angle value may be based on calculations of facial angle derived from digital images of the patient, or using other techniques. In accordance with some embodiments, the step of administering results in the subject having an increased G-Sn-Pog angle relative to the subject's G-Sn-Pog facial angle prior to the treatment, for example, immediately prior to the administering step. In some embodiments, the subject has an increased G-Sn-Pog angle for a period of time in the range of at least about 3 months, or more preferably, for at least about 6 months, for example, for about 9 months to about 24 months, after the step of administering. For example, the subject has an increased G-Sn-Pog angle for at least about 6 months, or for at least about 9 months, or for at least about 12 months or for at least about 18 months or for at least about 24 months after the step of administering.

In some embodiments, the treatment area is an area selected from the group consisting of the pogonion, the mentum, the left pre jowl sulcus, the right pre jowl sulcus, and the sublabial crease. The treatment may comprise administering the composition into two or more of the treatment areas. The administration comprises supraperiosteally or subdermally injecting the compositions in an amount of between about 0.5 mL and about 3.0 mL per treatment area. In some embodiments, the amount injected into a given treatment area is no greater than 2.0 mL. In some embodiments, the total amount injected in a single treatment session, over all treatment areas, is between 2.0 mL to about 6.0 mL, for example, about 2.5 mL, about 3.0 mL, about 3.5 mL, about 4.0 mL, about 4.5 mL, about 5.0 mL, about 5.5 mL, or about 6.0 mL. In some embodiments, the amount administered in a single treatment session is about 4.0 mL or less.

### Example 2

### Restoration and Creation of Volume in the Chin and Jaw

In one aspect, the present disclosure provides methods for restoring and creating volume in the chin and jaw, for example, in sculpting, shaping, and contouring across specific treatment areas of the face. The treatment areas may include one or more of the pogonion (the most projecting point on the anterior surface of the chin), mentum, (the lowest point on the chin), left and right pre-jowl sulci (left antigonion notch and right antigonion notch), and sublabial (mental) crease (the crease between the lower lip and the mentum).

The shape and projection of the chin contribute to the proportional balance of the face that underlies attractiveness. A chin lacking projection is commonly labeled a “weak chin” whereas prominent chins are labeled “strong chins” and imply strength of personality. Several studies have suggested that faces with average proportions are viewed as the most attractive and that juvenile features including a small chin are interpreted as attractive in females while a strong chin and jaw are interpreted as attractive in males. The appearance of the chin is a determinant of perceived attractiveness and can influence an individual's psychosocial well-being.

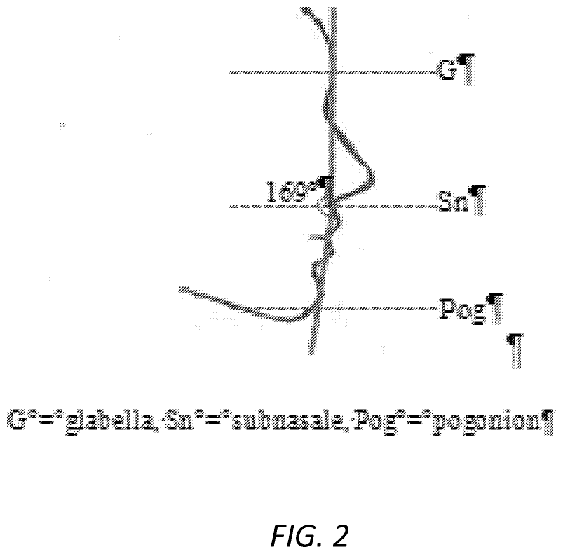

Average proportions are dictated by analysis of a representation of facial profiles in a population and include the distances and angles between the nose, lip, and mentum. Several soft tissue landmarks have been used in cephalometric analysis to measure and diagnose chin protrusion and retrusion deviations from average facial parameters. The intersection of the upper facial and anterior lower facial components and the angle formed by the point on the glabella, subnasale, and pogonion (G-Sn-Pog) has been extensively analyzed to understand the average chin projection common among populations. The Burstone angle (FIG. 2) has been defined as approximately 169° for the average chin, and the approximate angle (168° to 169°) has been confirmed in several studies.

Incrementally, deviations from the average chin result in the perception of facial unattractiveness. Analyzing the relationship between facial profile and perception of attractiveness shows that chin prominence plays a major role in this perception. To understand the relationship between the degree of chin prominence and attractiveness, a series of profile images altered in 2-mm increments from an idealized profile image was presented to a group of pretreatment orthognathic patients, clinicians, and laypeople. Subjects were asked to rate each image on a 7-point Likert scale ranging from extremely unattractive to extremely attractive. Ratings of perceived attractiveness decreased an average of 0.15 on the Likert Scale for each 2 mm of chin retrusion and were apparent after 4 mm of change. The degree of chin retrusion at which surgery was desired was 11 mm for patients and clinicians and 10 mm for laypeople. The most attractive image was that which displayed an ideal orthognathic profile with the soft tissue pogonion resting on the true vertical line.

### Example 3

### Method for Increasing the G-SN-POG Facial Angle in a Subject Having Chin Retrusion or a Weak Chin

A composition of the disclosure is administered as an injectable implant, by subdermal or supraperiosteal injection in the chin and/or jaw area of a 32 year old male subject. The subject complains he has a “weak chin.” The doctor measures the subject's facial angle and determines that the subject has a G-Sn-Pog angle of about 150°, which is substantially lower than the classic Burstone angle of the average chin (approximately 169°). The measurement is based on calculations of facial angle derived from digital images obtained using Canfield imaging equipment and software.

The doctor considers the subject's chin/jaw retrusion to be amenable to correction with a treatment goal consistent with increasing chin projection horizontally (in the profile view), not chin lengthening or widening.

The doctor believes that he can provide the subject with a more attractive facial profile and a stronger jawline by using the implantable compositions described herein.

The subject undergoes three treatment sessions, including initial treatment, top-up treatment, and repeat treatment, as described below.

For each treatment, the treatment areas include at least one or more of the following treatment areas: the pogonion (the most projecting point on the anterior surface of the chin), the mentum (the lowest point on the chin), the left pre-jowl sulcus (left antigonion notch), the right pre jowl sulcus (right antigonion notch), and/or the sublabial crease (the crease between the lower lip and the mentum).

The doctor implants no more than 2.0 mL into a single treatment area at any of the treatment sessions.

The initial treatment is performed on the subject as follows. The doctor uses aseptic skin preparation and administers anesthesia following his standard practice. The application of ice and topical anesthesia may reduce injection discomfort. Injectable anesthesia is limited to the treatment areas only and is administered with certainty not to distort the planned treatment areas.

Using needles (27 gauge×13 mm/27 G ½″) supplied with a kit, the doctor injects the compositions described herein subcutaneously and/or supraperiosteally to increase chin projection (horizontally in the profile view), as well as to aesthetically sculpt, contour, and shape, limiting treatment to the pogonion, mentum, pre jowl sulci, and sublabial (mental) crease. Suitable injection techniques have been described above. The treatment goal is to increase chin projection (horizontally in the profile view) and achieve aesthetic chin contour. The doctor determines the appropriate injection volume up to about 4.0 mL for initial and possible top-up treatments combined.

The doctor gently molds the treated area using manual manipulation of the overlying tissue to achieve the desired facial contour.

A top-up treatment occurs approximately 30 days after the initial treatment if desired by the subject, or if in the doctor's opinion, optimal (full) increase in chin projection and/or aesthetic contouring was not achieved by the initial treatment. If a top-up treatment is performed, the volume of the administered composition as a combined total (initial treatment and top-up treatment) is between about 2.0 mL to about 4.0 mL. During this visit, the doctor evaluates the treatment areas for any localized reaction and discusses any reported symptoms. 3D facial digital images (frontal and profile images) are captured for objective calculation of the angle of chin retrusion. If the doctor determines at top-up follow-up visit that optimal (full) increase in chin projection or aesthetic contouring was not achieved after the initial treatment, then subject is advised that he may receive a top-up treatment.

A single repeat treatment is administered at a scheduled visit between months 18 and 24 if repeat treatment is warranted in the doctor's opinion and/or is desired by the subject. Injection volume for the chin does not exceed a total volume of 4.0 mL for the repeat treatment.

### Example 4

### Method for Increasing the G-SN-POG Facial Angle in a Subject in Need Thereof

Subjects 18 years of age and older with a G-Sn-Pog facial angle of 145° to 165° received treatment with a composition disclosed herein administered as a dermal filler in order to improve their G-Sn-Pog facial angle. Subjects were randomized to either receive treatment at the onset of the study, or after a 3-month delay. The group that received treatment after a 3-month delay was used as the control group.

The subjects received treatment at up to 2 visits: an initial and a touch-up visit. The 2 visits were approximately 30 days apart.

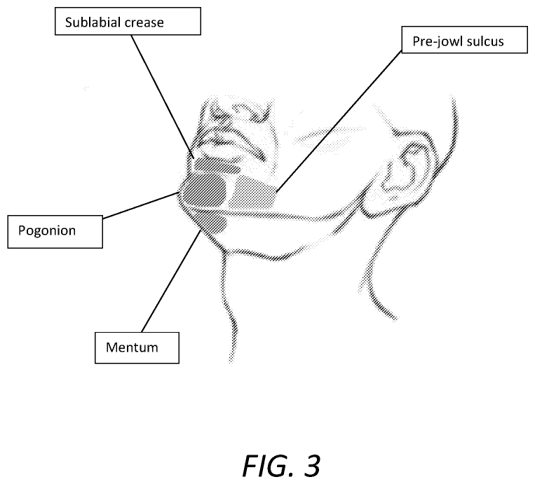

The volume of dermal filler used was limited to 2.0 mL per treatment session for any of the following regions: pogonion, mentum, left pre jowl sulcus, right pre jowl sulcus, and sublabial crease, as shown in FIG. 3. The dermal filler volume was further limited to a total of 4.0 mL combined for initial and touch-up treatments.

The subjects had follow-up visits 1 month, 3 months, 6 months, 9 months, and 12 months after the last treatment.

The subjects were assessed to compare the change in G-Sn-Pog facial angle from before treatment to after treatment. This assessment was done using 3-dimensional facial digital imaging, which was performed at all of the subjects' visits. A treatment-blinded image-analysis technician calculated all angle assessments performed based on glabella, subnasale, and pogonion landmarks that were set by the investigator in digital images that were captured at screening and each visit. These calculations were carried out by Canfield imaging software (Canfield Scientific, Inc., Parsippany, N.J., USA) using a built-in algorithm to calculate the G-Sn-Pog facial angle.

A total of 120 subjects were randomized for treatment. Ninety subjects were given the treatment immediately and 29 control subjects received treatment after 3 months. One subject in the control group discontinued participation before treatment. The baseline demographics and subject characteristics were similar in both groups and are set forth in Table 1 below.

TABLE 1

| | Treatment | | Control | | Total | |
|---|---|---|---|---|---|---|
| | (n = 90) | | (n = 30) | | (N = 120) | |
| Age, years | | | | | | |
| Mean (SD) | 46.4 | (12.87) | 45.7 | (14.67) | 46.2 | (13.28) |
| Median (range) | 48.5 | (22-75) | 50.0 | (20-68) | 49.0 | (20-75) |
| Female, n (%) | 81 | (90.0) | 29 | (96.7) | 110 | (91.7) |
| Race, n (%) | | | | | | |
| White | 74 | (82.2) | 29 | (96.7) | 103 | (85.8) |
| Black | 1 | (1.1) | 0 | | 1 | (0.8) |
| Asian descent | 14 | (15.6) | 1 | (3.3) | 15 | (12.5) |
| Other | 1 | (1.1) | 0 | | 1 | (0.8) |
| Body mass index, | 22.5 | (3.03) | 22.6 | (3.55) | 22.5 | (3.15) |
| mean (SD), kg/m² | | | | | | |
| Fitzpatrick skin | | | | | | |
| type, n (%) | | | | | | |
| I | 5 | (5.6) | 3 | (10.0) | 8 | (6.7) |
| II | 28 | (31.1) | 13 | (43.3) | 41 | (34.2) |
| III | 35 | (38.9) | 7 | (23.3) | 42 | (35.0) |
| IV | 17 | (18.9) | 6 | (20.0) | 23 | (19.2) |
| V | 4 | (4.4) | 1 | (3.3) | 5 | (4.2) |
| VI | 1 | (1.1) | 0 | | 1 | (0.8) |
| Baseline G-Sn-Pog | 160.6 | (4.2) | 161.3 | (2.8) | | |
| angle, mean (SD), | | | | | | |
| degrees | | | | | | |

The dermal filler was administered in accordance with the investigators' judgment and the parameters discussed above. The median injection volume of dermal filler for the initial and touch-up treatments combined was 3.43 mL (mean of 3.19 mL) in the treatment group and 3.55 mL (mean of 3.29 mL) in the control group. The mean and median injection volumes for initial and touch-up treatments combined by treatment area is set forth in Table 2 below.

TABLE 2

| | Treatment Group | Control (after treatment) |
|---|---|---|
| | Median (mean) Volume, | Median (mean) Volume, |
| Treatment Area | mL | mL |
| Sublabial crease | 0.45 (0.51) | 0.40 (0.47) |
| Pogonion | 0.90 (0.94) | 0.90 (0.86) |
| Mentum | 0.95 (0.93) | 1.25 (1.10) |
| Both pre-jowl sulci | 1.00 (1.05) | 1.00 (1.04) |

Nearly all subjects received initial treatment in the pogonion, mentum, and pre jowl sulci and 76% of subjects received initial treatment in the sublabial crease. Touch-up treatments were most commonly administered in the pogonion, pre-jowl sulci, and mentum.

The dermal filler was primarily administered using a serial puncture technique for initial and touch-up treatments. The most common plane of injection for the pogonion, mentum, and pre jowl sulci was supraperiosteal for both initial and touch-up treatments. For the sublabial crease, the majority of the injections were subcutaneous during initial treatment and subcutaneous or supraperiosteal during touch-up treatment.

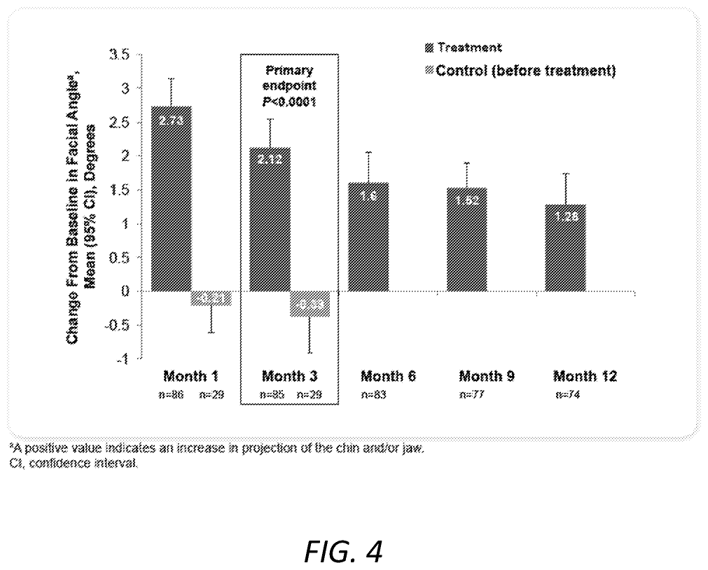

The mean increase in G-Sn-Pog facial angle from baseline was 2.12° at 3 months after treatment, as shown in FIG. 4. The mean increase in G-Sn-Pog facial angle for the control group after treatment was 1.96° at 3 months and 1.60° at 9 months. The subjects had a between-group difference in mean change in G-Sn-Pog facial angle from baseline to 3 months of 2.51° (95% Confidence Interval: 1.85°-3.17°; P<0.0001). The mean change from baseline in the G-Sn-Pog angle of the treatment group declined gradually over time, but remained 1.60° and 1.28° at 6 and 12 months, respectively. The mean change in G-Sn-Pog facial angle from baseline of the treatment group remained at 1.28° at 12 months.

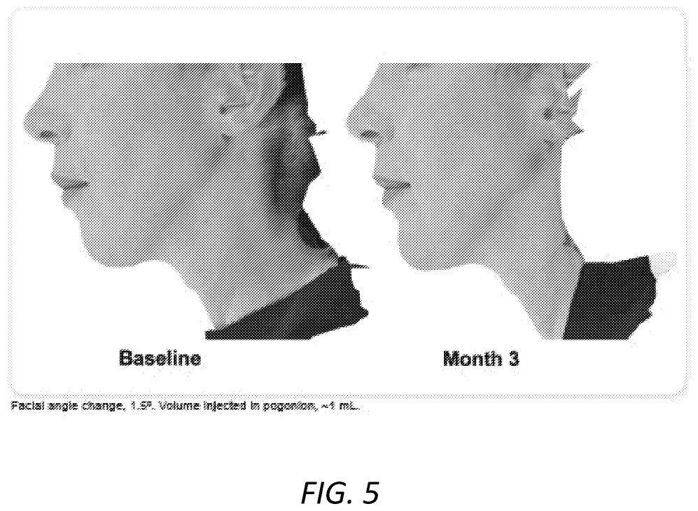

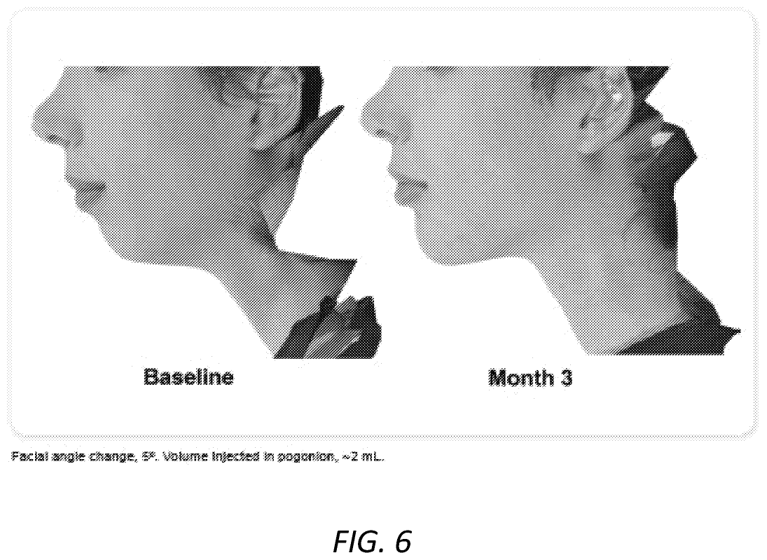

One subject had a G-Sn-Pog facial angle increase of 1.5° after approximately 1 mL of dermal filler was injected into the pogonion, as shown in FIG. 5. Another subject had a G-Sn-Pog facial angle increase of 5° after approximately 2 mL of dermal filler was injected into the pogonion, as shown in FIG. 6.

### Example 5

### Method for Increasing Chin Protrusion in a Subject in Need Thereof

Subjects 18 years of age and older received treatment with a composition of the disclosure administered as a dermal filler in order to improve their chin protrusion. Subjects were randomized to either receive treatment at the onset of the study, or after a 3-month delay. The group that received treatment after a 3-month delay was used as the control group.

The subjects received treatment at up to 2 visits: an initial and a touch-up visit. The 2 visits were approximately 30 days apart.

The investigators determined an appropriate treatment volume of the dermal filler. However, the volume of dermal filler used was limited to 2.0 mL per treatment session for any of the following regions: pogonion, mentum, left pre jowl sulcus, right pre jowl sulcus, and sublabial crease, as shown in FIG. 3. The dermal filler volume was further limited to a total of 4.0 mL combined for initial and touch-up treatments.

The subjects had follow-up visits 1 month, 3 months, 6 months, 9 months, and 12 months after the last treatment.

The subjects were assessed to compare the change in chin protrusion from before treatment to after treatment. This assessment was done using 3-dimensional facial digital imaging, which was performed at all of the subjects' visits. Calculations of chin protrusion were carried out by Canfield imaging software (Canfield Scientific, Inc., Parsippany, N.J., USA).

A total of 120 subjects were randomized for treatment. Ninety subjects were given the treatment immediately and 29 control subjects received treatment after 3 months. One subject in the control group discontinued participation before treatment. The baseline demographics and subject characteristics were similar in both groups and are set forth in Table 3 below.

TABLE 3

| | Treatment | | Control | | Total | |
|---|---|---|---|---|---|---|
| | (n = 90) | | (n = 30) | | (N = 120) | |
| Age, years | | | | | | |
| Mean (SD) | 46.4 | (12.87) | 45.7 | (14.67) | 46.2 | (13.28) |
| Median (range) | 48.5 | (22-75) | 50.0 | (20-68) | 49.0 | (20-75) |
| Female, n (%) | 81 | (90.0) | 29 | (96.7) | 110 | (91.7) |
| Race, n (%) | | | | | | |
| White | 74 | (82.2) | 29 | (96.7) | 103 | (85.8) |
| Black | 1 | (1.1) | 0 | | 1 | (0.8) |
| Asian descent | 14 | (15.6) | 1 | (3.3) | 15 | (12.5) |
| Other | 1 | (1.1) | 0 | | 1 | (0.8) |
| Body mass index, | 22.5 | (3.03) | 22.6 | (3.55) | 22.5 | (3.15) |
| mean (SD), kg/m² | | | | | | |
| Fitzpatrick skin | | | | | | |
| type, n (%) | | | | | | |
| I | 5 | (5.6) | 3 | (10.0) | 8 | (6.7) |
| II | 28 | (31.1) | 13 | (43.3) | 41 | (34.2) |
| III | 35 | (38.9) | 7 | (23.3) | 42 | (35.0) |
| IV | 17 | (18.9) | 6 | (20.0) | 23 | (19.2) |
| V | 4 | (4.4) | 1 | (3.3) | 5 | (4.2) |

| | | | | | |
|---|---|---|---|---|---|
| VI | 1 | (1.1) | 0 | 1 | (0.8) |

The dermal filler was administered in accordance with the investigators' judgment and the parameters discussed above. The median injection volume of dermal filler for the initial and touch-up treatments combined was 3.43 mL (mean of 3.19 mL) in the treatment group and 3.55 mL (mean of 3.29 mL) in the control group. The mean and median injection volumes for initial and touch-up treatments combined by treatment area is set forth in Table 4 below.

TABLE 4

| | Treatment Group | Control (after treatment) |
|---|---|---|
| | Median (mean) Volume, | Median (mean) Volume, |
| Treatment Area | mL | mL |
| Sublabial crease | 0.45 (0.51) | 0.40 (0.47) |
| Pogonion | 0.90 (0.94) | 0.90 (0.86) |
| Mentum | 0.95 (0.93) | 1.25 (1.10) |
| Both pre-jowl sulci | 1.00 (1.05) | 1.00 (1.04) |

Nearly all subjects received initial treatment in the pogonion, mentum, and pre jowl sulci and 76% of subjects received initial treatment in the sublabial crease. Touch-up treatments were most commonly administered in the pogonion, pre-jowl sulci, and mentum.

The dermal filler was primarily administered using a serial puncture technique for initial and touch-up treatments. The most common plane of injection for the pogonion, mentum, and pre jowl sulci was supraperiosteal for both initial and touch-up treatments. For the sublabial crease, the majority of the injections were subcutaneous during initial treatment and subcutaneous or supraperiosteal during touch-up treatment.

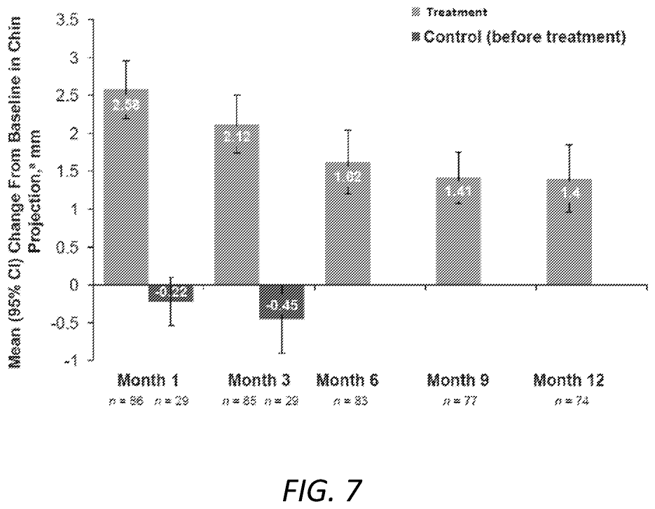

The mean change from baseline in chin protrusion in the treatment versus control subjects was 2.12 mm at 3 months versus a decrease of 0.45 mm in the control group before treatment at 3 months, as shown in FIG. 7. The increase in chin protrusion decreased gradually through 12 months after treatment, but remained above baseline values. At 12 months, there was still an increase in chin protrusion of 1.4 mm.

### Example 6

### Method of Treating Chin Retrusion in a Subject by Increasing the G-SN-POG Facial Angle of the Subject

Subjects 18 years of age and older with a G-Sn-Pog facial angle of 145° to 165° received treatment with a composition disclosed herein administered as a dermal filler in order to treat chin retrusion by improving their G-Sn-Pog facial angle. Subjects were randomized to either receive treatment at the onset of the study, or after a 3-month delay. The group that received treatment after a 3-month delay was used as the control group.

The subjects received treatment at up to 2 visits: an initial and a touch-up visit. The 2 visits were approximately 30 days apart.

The volume of dermal filler used was limited to 2.0 mL per treatment session for any of the following regions: pogonion, mentum, left pre jowl sulcus, right pre jowl sulcus, and sublabial crease, as shown in FIG. 3. The dermal filler volume was further limited to a total of 4.0 mL combined for initial and touch-up treatments.

The subjects had follow-up visits 1 month, 3 months, 6 months, 9 months, and 12 months after the last treatment.

The subjects were assessed to compare the change in G-Sn-Pog facial angle from before treatment to after treatment. This assessment was done using 3-dimensional facial digital imaging, which was performed at all of the subjects' visits. A treatment-blinded image-analysis technician calculated all angle assessments performed based on glabella, subnasale, and pogonion landmarks that were set by the investigator in digital images that were captured at screening and each visit. These calculations were carried out by Canfield imaging software (Canfield Scientific, Inc., Parsippany, N.J., USA) using a built-in algorithm to calculate the G-Sn-Pog facial angle.

A total of 120 subjects were randomized for treatment. Ninety subjects were given the treatment immediately and 29 control subjects received treatment after 3 months. One subject in the control group discontinued participation before treatment. The baseline demographics and subject characteristics were similar in both groups and are set forth in Table 5 below.

TABLE 5

| | Treatment | | Control | | Total | |
|---|---|---|---|---|---|---|
| | (n = 90) | | (n = 30) | | (N = 120) | |
| Age, years | | | | | | |
| Mean (SD) | 46.4 | (12.87) | 45.7 | (14.67) | 46.2 | (13.28) |
| Median (range) | 48.5 | (22-75) | 50.0 | (20-68) | 49.0 | (20-75) |
| Female, n (%) | 81 | (90.0) | 29 | (96.7) | 110 | (91.7) |
| Race, n (%) | | | | | | |
| White | 74 | (82.2) | 29 | (96.7) | 103 | (85.8) |
| Black | 1 | (1.1) | 0 | | 1 | (0.8) |
| Asian descent | 14 | (15.6) | 1 | (3.3) | 15 | (12.5) |
| Other | 1 | (1.1) | 0 | | 1 | (0.8) |
| Body mass index, | 22.5 | (3.03) | 22.6 | (3.55) | 22.5 | (3.15) |
| mean (SD), kg/m² | | | | | | |
| Fitzpatrick skin | | | | | | |
| type, n (%) | | | | | | |
| I | 5 | (5.6) | 3 | (10.0) | 8 | (6.7) |
| II | 28 | (31.1) | 13 | (43.3) | 41 | (34.2) |
| III | 35 | (38.9) | 7 | (23.3) | 42 | (35.0) |
| IV | 17 | (18.9) | 6 | (20.0) | 23 | (19.2) |
| V | 4 | (4.4) | 1 | (3.3) | 5 | (4.2) |
| VI | 1 | (1.1) | 0 | | 1 | (0.8) |
| Baseline G-Sn-Pog | 160.6 | (4.2) | 161.3 | (2.8) | | |
| angle, mean (SD), | | | | | | |
| degrees | | | | | | |

The dermal filler was administered in accordance with the investigators' judgment and the parameters discussed above. The median injection volume of dermal filler for the initial and touch-up treatments combined was 3.43 mL (mean of 3.19 mL) in the treatment group and 3.55 mL (mean of 3.29 mL) in the control group. The mean and median injection volumes for initial and touch-up treatments combined by treatment area is set forth in Table 6 below.

TABLE 6

| | Treatment Group | Control (after treatment) |
|---|---|---|
| | Median (mean) Volume, | Median (mean) Volume, |
| Treatment Area | mL | mL |
| Sublabial crease | 0.45 (0.51) | 0.40 (0.47) |
| Pogonion | 0.90 (0.94) | 0.90 (0.86) |
| Mentum | 0.95 (0.93) | 1.25 (1.10) |
| Both pre-jowl sulci | 1.00 (1.05) | 1.00 (1.04) |

Nearly all subjects received initial treatment in the pogonion, mentum, and pre jowl sulci and 74.4% of treatment subjects and 79.3% of control subjects received treatment in the sublabial crease. For example, touch-up treatments were administered in the pogonion, pre-jowl sulci, and mentum. The median and range of injection volumes provided to the treatment and control groups as well as the number of subjects receiving injections in each location are set forth in Table 7 below.

TABLE 7

| | |
|---|---|
| Treatment Group | Control Group |
| (n = 90) Median | (n = 29) Median |
| (range) Volume, mL | (range) Volume, mL |

Total Volume

| | | | | |
|---|---|---|---|---|
| Initial/touch-up | n = 90 | (100%) | n = 29 | (100%) |
| treatment combined | 3.43 mL | (1.2-4.1 mL) | 3.55 mL | (1.7-4.0 mL) |
| Initial treatment | n = 90 | (100%) | n = 29 | (100%) |
| | 2.28 mL | (1.0-4.0 mL) | 2.50 mL | (1.6-3.0 mL) |
| Touch-up treatment | n = 69 | (76.7%) | n = 22 | (75.9%) |
| | 1.00 mL | (0.2-2.0 mL) | 1.00 mL | (0.3-2.0 mL) |

Initial/Touch-Up Treatment Combined

| | | | | |
|---|---|---|---|---|
| Pogonion | n = 90 | (100%) | n = 29 | (100%) |
| | 0.90 mL | (0.1-2.1 mL) | 0.90 mL | (0.1-1.9 mL) |
| Mentum | n = 85 | (94.4%) | n = 27 | (93.1%) |
| | 0.95 mL | (0.1-2.1 mL) | 1.25 mL | (0.2-2.2 mL) |
| Prejowl sulci - | n = 85 | (94.4%) | n = 29 | (100%) |
| combined right | 1.00 mL | (0.2-3.0 mL) | 1.00 mL | (0.2-2.4 mL) |
| and left | | | | |
| Sublabial crease | n = 67 | (74.4%) | n = 23 | (79.3%) |
| | 0.45 mL | (0.1-1.2 mL) | 0.40 mL | (0.1-1.2 mL) |

For instance, the dermal filler was administered using a serial puncture technique for initial (75.9% of the treatment group) and touch-up treatments (74.1% of the treatment group). Also, for example, the plane of injection for the pogonion, mentum, and pre jowl sulci was supraperiosteal for both initial and touch-up treatments. The injections for the sublabial crease, for instance, were subcutaneous during initial treatment (60.3% of the treatment group) and subcutaneous or supraperiosteal during touch-up treatment (41.9% and 45.2% of the treatment group, respectively). The injection techniques and planes of injections used are shown in Table 8 below. Subjects could have received an injection in more than 1 plane and could have received more than 1 injection technique.

TABLE 8

| | Treatment Group | | | | Control Group | | | |
|---|---|---|---|---|---|---|---|---|
| | Initial | | Touch-up | | Initial | | Touch-up | |
| Pogonion | n = 87 | | n = 54 | | n = 29 | | n = 16 | |
| Plane of injection, n (%) | | | | | | | | |
| Supraperiosteal only | 64 | (73.6) | 42 | (77.8) | 20 | (69.0) | 11 | (68.8) |
| Subcutaneous only | 16 | (18.4) | 12 | (22.2) | 6 | (20.7) | 4 | (25.0) |
| Subcutaneous and | 7 | (8.0) | 0 | | 3 | (10.3) | 1 | (6.3) |
| supraperiosteal | | | | | | | | |
| Injection technique, n (%) | | | | | | | | |
| Serial puncture | 66 | (75.9) | 40 | (74.1) | 20 | (69.0) | 13 | (81.3) |
| Tunneling | 19 | (21.8) | 13 | (24.1) | 8 | (27.6) | 3 | (18.8) |
| Fanning | 2 | (2.3) | 1 | (1.9) | 1 | (3.4) | 0 | |
| Mentum | n = 85 | | n = 40 | | n = 27 | | n = 14 | |
| Plane of injection, n (%) | | | | | | | | |
| Supraperiosteal only | 70 | (82.4) | 36 | (90.0) | 21 | (77.8) | 12 | (85.7) |
| Subcutaneous only | 11 | (12.9) | 3 | (7.5) | 4 | (14.8) | 2 | (14.3) |
| Subcutaneous and | 4 | (4.7) | 1 | (2.5) | 2 | (7.4) | 0 | |
| supraperiosteal | | | | | | | | |
| Injection technique, n (%) | | | | | | | | |
| Serial puncture | 64 | (75.3) | 33 | (82.5) | 20 | (74.1) | 12 | (85.7) |
| Tunneling | 21 | (24.7) | 7 | (17.5) | 7 | (25.9) | 2 | (14.3) |
| Fanning | 1 | (1.2) | 0 | | 1 | (3.7) | 0 | |
| Prejowl sulcus, left | n = 81 | | n = 52 | | n = 28 | | n = 15 | |
| Plane of injection, n (%) | | | | | | | | |
| Supraperiosteal only | 56 | (69.1) | 35 | (67.3) | 19 | (67.9) | 7 | (46.7) |
| Subcutaneous only | 22 | (27.2) | 16 | (30.8) | 8 | (28.6) | 8 | (53.3) |
| Subcutaneous and | 3 | (3.7) | 1 | (1.9) | 1 | (3.6) | 0 | |
| supraperiosteal | | | | | | | | |
| Other | 0 | | 0 | | 0 | | 0 | |
| Injection technique, n (%) | | | | | | | | |
| Serial puncture | 47 | (58.0) | 34 | (65.4) | 18 | (64.3) | 9 | (60.0) |
| Tunneling | 28 | (34.6) | 14 | (26.9) | 7 | (25.0) | 2 | (13.3) |
| Fanning | 6 | (7.4) | 6 | (11.5) | 5 | (17.9) | 4 | (26.7) |
| Prejowl sulcus, right | n = 82 | | n = 51 | | n = 28 | | n = 15 | |
| Plane of injection, n (%) | | | | | | | | |
| Supraperiosteal only | 56 | (68.3) | 32 | (62.7) | 19 | (67.9) | 7 | (46.7) |
| Subcutaneous only | 23 | (28.0) | 18 | (35.3) | 8 | (28.6) | 8 | (53.3) |
| Subcutaneous and | 3 | (3.7) | 1 | (2.0) | 1 | (3.6) | 0 | |
| supraperiosteal | | | | | | | | |
| Other | 0 | | 0 | | 0 | | 0 | |
| Injection technique, n (%) | | | | | | | | |
| Serial puncture | 48 | (58.5) | 34 | (66.7) | 18 | (64.3) | 9 | (60.0) |
| Tunneling | 28 | (34.1) | 13 | (25.5) | 7 | (25.0) | 2 | (13.3) |
| Fanning | 6 | (7.3) | 6 | (11.8) | 5 | (17.9) | 4 | (26.7) |
| Sublabial crease | n = 63 | | n = 31 | | n = 22 | | n = 4 | |
| Plane of injection, n (%) | | | | | | | | |
| Supraperiosteal only | 38 | (60.3) | 13 | (41.9) | 15 | (68.2) | 1 | (25.0) |
| Subcutaneous only | 16 | (25.4) | 14 | (45.2) | 6 | (27.3) | 3 | (75.0) |
| Subcutaneous and | 1 | (1.6) | 0 | | 1 | (4.5) | 0 | |
| supraperiosteal | | | | | | | | |
| Other | 8 | (12.7) | 4 | (12.9) | 0 | | 0 | |
| Injection technique, n (%) | | | | | | | | |
| Serial puncture | 31 | (49.2) | 18 | (58.1) | 9 | (40.9) | 3 | (75.0) |
| Tunneling | 24 | (38.1) | 11 | (35.5) | 12 | (54.5) | 1 | (25.0) |

| | | | | | | | |
|---|---|---|---|---|---|---|---|
| Fanning | 8 | (12.7) | 3 | (9.7) | 1 | (4.5) | 0 |

| | | | | | |
|---|---|---|---|---|---|
| Crosshatching | 2 | (3.2) | 0 | 0 | 0 |

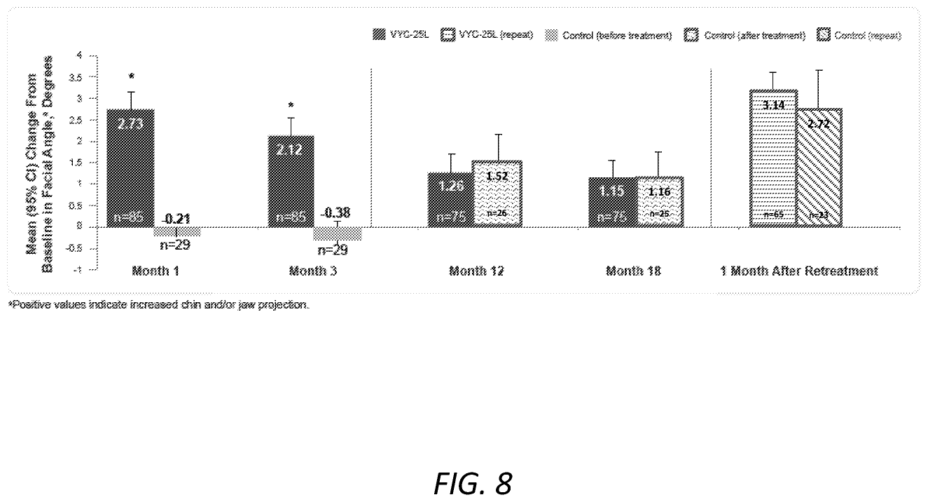

The mean increase in G-Sn-Pog facial angle from baseline was 2.73° at 1 month after treatment and 2.12° at 3 months after treatment, as shown in FIG. 8. The subjects had a between-group difference in mean change in G-Sn-Pog facial angle from baseline to 3 months of 2.51° (95% Confidence Interval: 1.85°-3.17°; P<0.0001). The mean change from baseline to 1 month after repeat treatment was 3.14°, also shown in FIG. 8.

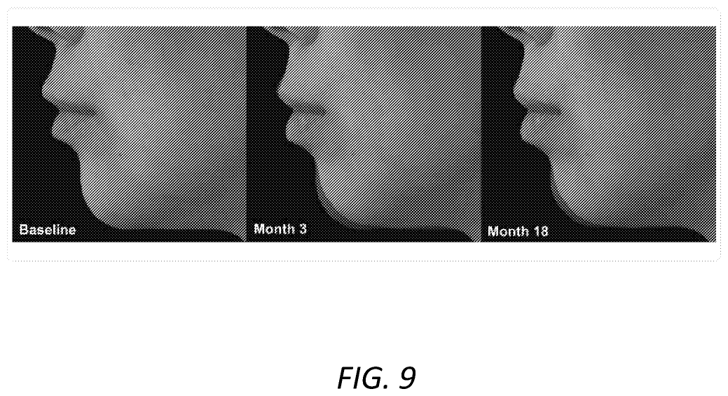

The mean change from baseline in the G-Sn-Pog angle of the treatment group declined gradually over time, but remained 1.26° at 12 months after treatment and 1.15° after 18 months after treatment, as shown in FIG. 8. Representative images of a subject treated with the dermal filler at baseline, 3 months after treatment, and 18 months after treatment are shown in FIG. 9.

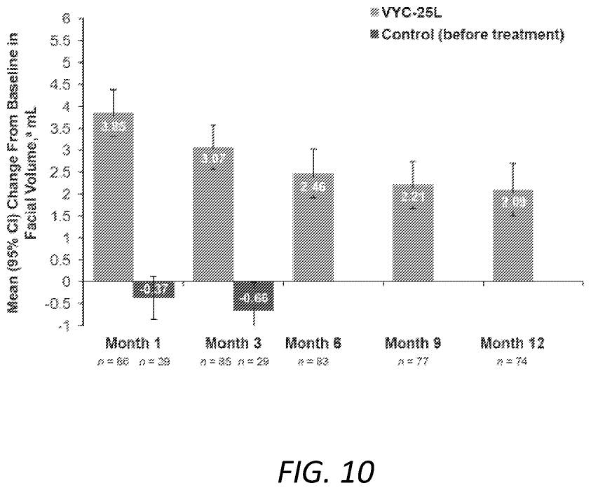

Analysis of three-dimensional facial images showed that the mean change from baseline to 3 months after treatment in facial volume was 3.07 mL in the treatment group compared to a decrease of 0.66 mL in the control group before treatment, as shown in FIG. 10. The improvements to facial volume decreased gradually through 12 months after treatment, but remained above baseline values.

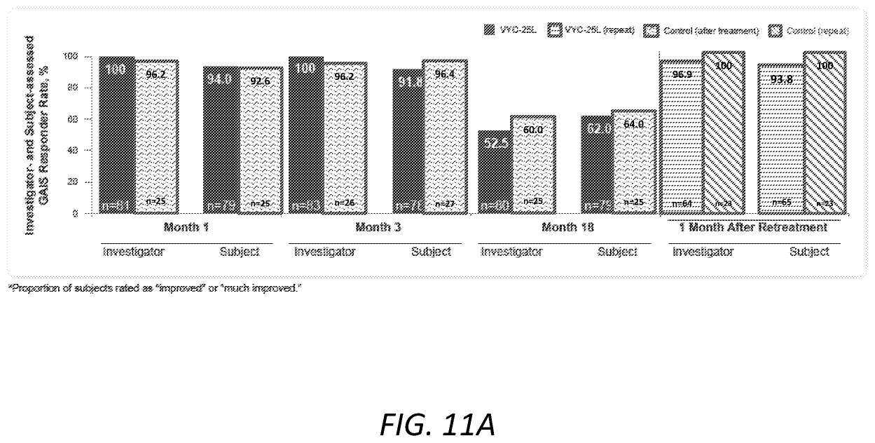

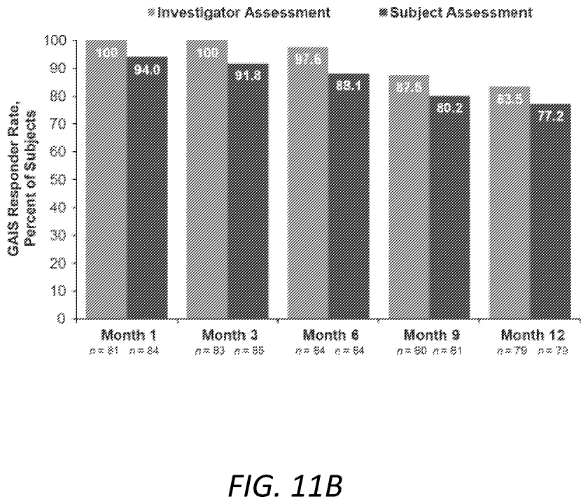

Global aesthetic improvement in the chin and jaw area was assessed as a secondary effectiveness end point by both investigators and subjects at 1 month, 3 months, 6 months, 9 months, 12 months, and 18 months after treatment using the 5-point Global Aesthetic Improvement Scale (GAIS). Scores ranged from −2 (much worse) to +2 (much improved). GAIS responder rates based on investigator and subject assessments were 100% and 91.8%, respectively, at 3 months after treatment and 83.5% and 77.2%, respectively, at 12 months after treatment. GAIS responder rates based on investigator and subject assessment are shown in FIGS. 11A and 11B. Even 18 months after treatment, investigator scores remained above 50%. GAIS responder rates increased again to 96.9% for investigator assessment of the treatment group 1 month after touch-up or re-treatment.

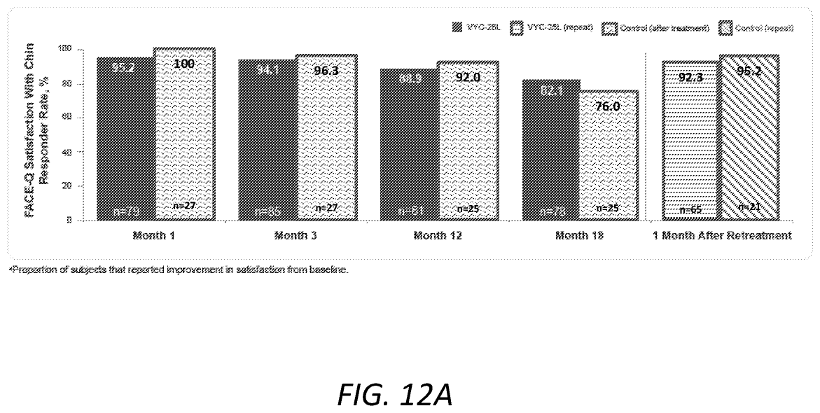

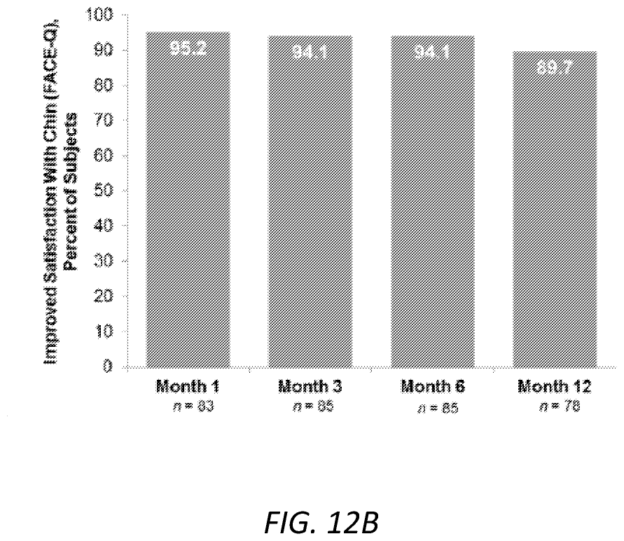

Effectiveness was assessed by subject responses on three FACE-Q scales, satisfaction with chin, satisfaction with lower face and jawline, and psychological well-being, at 1 month, 3 months, 6 months, 12 months, and 18 months after treatment. Mean scores on the FACE-Q satisfaction with chin improved from 41.1% at baseline to 72.8% at 3 months after treatment and 61.6% 12 months after treatment. For example, 95.2% of subjects in the treatment group reported improved satisfaction with their chin at 1 month after treatment, as shown in FIGS. 12A and 12B. Satisfaction declined slightly over time, but was maintained through 18 months at 82.1%. Responder rates increased again after re-treatment to 92.3%.

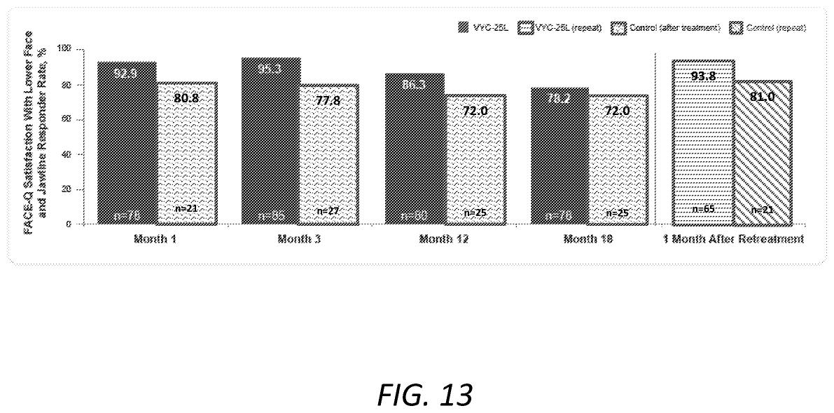

The mean score on the FACE-Q satisfaction with lower face and jawline increased from 36.7% at baseline to 70.2% at 3 months after treatment and 60.6% at 12 months after treatment. For example, subjects in the treatment group reported improved satisfaction with their lower face and jaw, which declined gradually from 95.3% at 3 months after treatment, to 87.0% at 12 months after treatment, to 78.2% at 18 months after treatment, as shown in part in FIG. 13. Responder rates increased after re-treatment (93.8%).

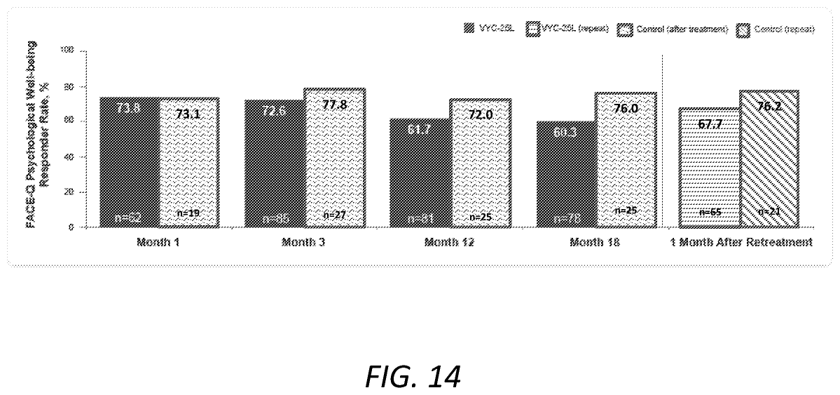

Additionally, the mean score on the FACE-Q with psychological well-being improved from 65.3% at baseline to 78.7% at 3 months after treatment and 74.4% at 12 months after treatment. For example, 73.8% of subjects in the treatment group reported improved psychological well-being at 1 month after treatment, which remained at 18 months after treatment (60.3%), as shown in FIG. 14. When assessed 1 month after re-treatment, responder rates increased to 67.7%. Similar responder rates were reported by the control group.

Investigators also used an 11-point scale to rate the ease of injection (0=difficult, 10=easy) and product moldability (0=stiff, 10=moldable) after the initial and touch-up treatments. In the treatment group, the mean scores for ease of injection were 8.7 for initial treatment and 9.1 for touch-up treatment. Mean scores for moldability were 8.2 after initial treatment and 8.6 after touch-up treatment. Comparable ease of injection and moldabilty scores were reported in the control group.

The subjects also rated their pain immediately after the initial and touch-up treatments on an 11-point scale (0=no pain, 10=worst pain imaginable), and used a safety diary for 30 days after each treatment to record the presence, severity, and duration of any injection site responses (ISRs). The subjects also completed the recovery early life impact scale and recovery early symptoms checklist of FACE-Q 3 days after the initial treatment. Adverse events were also monitored at each visit.

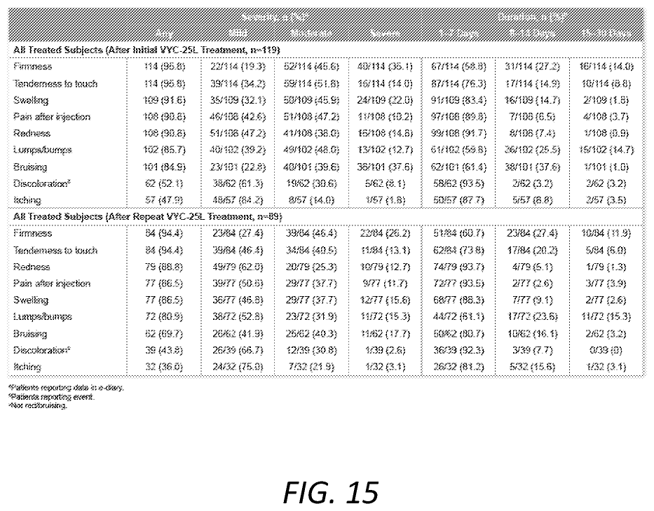

Mean pain scores assessed immediately after treatment for all treated subjects were 2.2 and 2.1 after initial and touch-up treatments, respectively. For example, 115/119 or 96.6% of subjects reported at least 1 ISR in any treatment area after initial treatment and 95.5% reported at least 1 ISR after repeat treatment, as shown in FIG. 15. The most frequently reported ISRs were firmness, tenderness to touch, and swelling, as shown in FIG. 15. Most ISRs were mild or moderate in severity and resolved within 1 week after treatment.

The mean overall score on the FACE-Q recovery early life impact scale on day 3 after treatment was 76.1% in the treatment group and 74.2% in the control group. For example, 71.9% of the subjects in the treatment group and 75.9% of the subjects in the control group reported performing usual daily activities within the first 3 days after treatment. On the recovery early symptoms questionnaire, subjects reported feeling not at all or a little bothered by symptoms on day 3 after treatment.

Treatment-related adverse events (AEs) occurred in 46 subjects (38.7%) after initial treatment. These subjects had 98 treatment-related AEs, most of which occurred at the injection site (92.9%). Of the treatment-related AEs, 1.7% of subjects reported headache and 3.4% reported minor speech disorder (specifically “unclear pronunciation”), all of which were transient. Most AEs resolved without treatment. After repeat treatment, 18 subjects (20.2%) had 53 treatment-related AEs, all at the injection site. For example, AEs for both initial and repeat treatments included injection site mass, induration, and/or pain. There were no treatment-related serious AEs, deaths, or AEs that led to study discontinuation. Treatment-emergent AEs (TEAEs) that occurred are set forth in Table 9 below.

TABLE 9

| Adverse Event, n | All Treated Subjects | Events | |
|---|---|---|---|
| (%) | (n = 119) | (n = 45) | |
| Any treatment-related TEAE | 30 (25.2%) | 45 | (100.0%) |
| Treatment-related TEAE at | 25 (21.0%) | 36 | (80.0%) |
| injection site | | | |
| Injection site mass | 10 (8.4%) | 10 | (22.2%) |
| Injection site pain | 6 (5.0%) | 9 | (20.0%) |
| Injection site swelling | 4 (3.4%) | 4 | (8.9%) |
| Speech disorder | 4 (3.4%) | 6 | (13.3%) |
| Injection site induration | 3 (2.5%) | 3 | (6.7%) |
| Headache | 2 (1.7%) | 2 | (4.4%) |
| Injection site nodule | 2 (1.7%) | 2 | (4.4%) |
| Injection site bruising | 1 (0.8%) | 1 | (2.2%) |
| Injection site cyst | 1 (0.8%) | 1 | (2.2%) |
| Injection site discoloration | 1 (0.8%) | 1 | (2.2%) |
| Injection site erythema | 1 (0.8%) | 1 | (2.2%) |
| Injection site pruritus | 1 (0.8%) | 1 | (2.2%) |
| Presyncope | 1 (0.8%) | 1 | (2.2%) |
| Myalgia | 1 (0.8%) | 1 | (2.2%) |
| Device dislocation | 1 (0.8%) | 1 | (2.2%) |
| Skin tightness | 1 (0.8%) | 1 | (2.2%) |

### Example 7

### Method for Treating Chin Retrusion in a Subject in Need Thereof

Subjects 18 years of age and older received treatment with a composition of the disclosure administered as a dermal filler in order to treat their chin retrusion. Subjects were randomized to either receive treatment at the onset of the study, or after a 3-month delay. The group that received treatment after a 3-month delay was used as the control group.

The subjects received treatment at up to 2 visits: an initial and a touch-up visit. The 2 visits were approximately 30 days apart.

The investigators determined an appropriate treatment volume of the dermal filler. However, the volume of dermal filler used was limited to 2.0 mL per treatment session for any of the following regions: pogonion, mentum, left pre jowl sulcus, right pre jowl sulcus, and sublabial crease, as shown in FIG. 3. The dermal filler volume was further limited to a total of 4.0 mL combined for initial and touch-up treatments.

The subjects had follow-up visits 1 month, 3 months, 6 months, 9 months, and 12 months after the last treatment.

The subjects were assessed to compare the change in chin protrusion from before treatment to after treatment. This assessment was done using 3-dimensional facial digital imaging, which was performed at all of the subjects' visits. Calculations of chin protrusion were carried out by Canfield imaging software (Canfield Scientific, Inc., Parsippany, N.J., USA).

A total of 120 subjects were randomized for treatment. Ninety subjects were given the treatment immediately and 29 control subjects received treatment after 3 months. One subject in the control group discontinued participation before treatment. The baseline demographics and subject characteristics were similar in both groups and are set forth in Table 10 below.

TABLE 10

| | Treatment | | Control | | Total | |
|---|---|---|---|---|---|---|
| | (n = 90) | | (n = 30) | | (N = 120) | |
| Age, years | | | | | | |
| Mean (SD) | 46.4 | (12.87) | 45.7 | (14.67) | 46.2 | (13.28) |
| Median (range) | 48.5 | (22-75) | 50.0 | (20-68) | 49.0 | (20-75) |
| Female, n (%) | 81 | (90.0) | 29 | (96.7) | 110 | (91.7) |
| Race, n (%) | | | | | | |
| White | 74 | (82.2) | 29 | (96.7) | 103 | (85.8) |
| Black | 1 | (1.1) | 0 | | 1 | (0.8) |
| Asian descent | 14 | (15.6) | 1 | (3.3) | 15 | (12.5) |
| Other | 1 | (1.1) | 0 | | 1 | (0.8) |
| Body mass index, | 22.5 | (3.03) | 22.6 | (3.55) | 22.5 | (3.15) |
| mean (SD), kg/m² | | | | | | |
| Fitzpatrick skin | | | | | | |
| type, n (%) | | | | | | |
| I | 5 | (5.6) | 3 | (10.0) | 8 | (6.7) |
| II | 28 | (31.1) | 13 | (43.3) | 41 | (34.2) |
| III | 35 | (38.9) | 7 | (23.3) | 42 | (35.0) |
| IV | 17 | (18.9) | 6 | (20.0) | 23 | (19.2) |
| V | 4 | (4.4) | 1 | (3.3) | 5 | (4.2) |

| | | | | | |
|---|---|---|---|---|---|
| VI | 1 | (1.1) | 0 | 1 | (0.8) |

The dermal filler was administered in accordance with the investigators' judgment and the parameters discussed above. The median injection volume of dermal filler for the initial and touch-up treatments combined was 3.43 mL (mean of 3.19 mL) in the treatment group and 3.55 mL (mean of 3.29 mL) in the control group. The mean and median injection volumes for initial and touch-up treatments combined by treatment area is set forth in Table 11 below.

TABLE 11

| | Treatment Group | Control (after treatment) |
|---|---|---|
| | Median (mean) Volume, | Median (mean) Volume, |
| Treatment Area | mL | mL |
| Sublabial crease | 0.45 (0.51) | 0.40 (0.47) |
| Pogonion | 0.90 (0.94) | 0.90 (0.86) |
| Mentum | 0.95 (0.93) | 1.25 (1.10) |
| Both pre-jowl sulci | 1.00 (1.05) | 1.00 (1.04) |

Nearly all subjects received initial treatment in the pogonion, mentum, and pre jowl sulci and 76% of subjects received initial treatment in the sublabial crease. For example, touch-up treatments were administered in the pogonion, pre jowl sulci, and mentum.

For example, the dermal filler was administered using a serial puncture technique for initial and touch-up treatments. Also, the injections of the dermal filler for the pogonion, mentum, and pre jowl sulci were, for instance, supraperiosteal for both initial and touch-up treatments. For the sublabial crease, the injections were, for example, subcutaneous during initial treatment and subcutaneous or supraperiosteal during touch-up treatment.

The mean change from baseline in chin retrusion in the treatment versus control subjects was a decrease in chin retrusion 2.12 mm at 3 months versus an increase in chin retrusion of 0.45 mm in the control group before treatment at 3 months. The decrease in chin retrusion gradually lessened through 12 months after treatment, but remained improved over baseline values. At 12 months, there was still a decrease in chin retrusion of 1.4 mm.

Although the disclosure has been described and illustrated with a certain degree of particularity, it is understood that the present disclosure has been made only by way of example, and that numerous changes in the combination and arrangement of parts can be resorted to by those skilled in the art without departing from the scope of the disclosure, as hereinafter claimed.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present disclosure. At the very least, and not as an attempt to limit the application of the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the disclosure are approximations, the numerical values set forth in the specific examples are reported as precisely as possible. Any numerical value, however, inherently contains certain errors necessarily resulting from the standard deviation found in their respective testing measurements. Notwithstanding that the numerical ranges and parameters setting forth the broad scope of the disclosure are approximations, the numerical values set forth in the specific examples are reported as precisely as possible. Any numerical value, however, inherently contains certain errors necessarily resulting from the standard deviation found in their respective testing measurements.

Specific embodiments disclosed herein may be further limited in the claims using consisting of or consisting essentially of language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the disclosure so claimed are inherently or expressly described and enabled herein.

In closing, it is to be understood that the embodiments of the disclosure disclosed herein are illustrative of the principles of the present disclosure. Other modifications that may be employed are within the scope of the disclosure. Thus, by way of example, but not of limitation, alternative configurations of the present disclosure may be utilized in accordance with the teachings herein. Accordingly, the present disclosure is not limited to that precisely as shown and described.

1. A method of treating chin retrusion in a subject by increasing a glabella-subnasale-pogonion (G-Sn-Pog) facial angle of the subject, the method comprising:
injecting a first volume of a hyaluronic acid (HA)-based dermal filler into a pogonion;
injecting a second volume of the HA-based dermal filler into a mentum; and
injecting a third volume of the HA-based dermal filler into a left pre-jowl sulcus and a right pre-jowl sulcus,
wherein the G-Sn-Pog facial angle of the subject is increased after injection of the HA-based dermal filler into the pogonion, mentum, left pre jowl sulcus, and right pre jowl sulcus by at least about 1°, and
wherein the HA-based dermal filler composition comprises hyaluronic acid (HA) crosslinked with 1,4-butanediol diglycidyl ether (BDDE);
wherein a HA concentration of the composition is greater than 20 mg/g;
wherein the HA used for crosslinking is made with a mixture of low molecular weight hyaluronic acid and high molecular weight hyaluronic acid; and
wherein the HA used for crosslinking is a mixture containing at least 50% by weight of low molecular weight HA, based on a total weight of the HA.

2. The method of claim 1, wherein the increase in the G-Sn-Pog facial angle of the subject is maintained for at least about 3 months.

3. The method of claim 1, wherein the increase in the G-Sn-Pog facial angle of the subject is maintained for at least about 12 months.

4. The method of claim 1, wherein the increase in the G-Sn-Pog facial angle of the subject is maintained for at least about 18 months.

5. The method of claim 1, wherein the G-Sn-Pog facial angle of the subject is increased by at least about 2°.

6. The method of claim 5, wherein an increase in the G-Sn-Pog facial angle of the subject of at least about 2° is maintained for at least about 18 months.

7. The method of claim 1, wherein the injection of the HA-based dermal filler into the pogonion, mentum, left pre jowl sulcus, and right pre jowl sulcus occurs at a first treatment session.

8. The method of claim 7 further comprising a second treatment session about 18 months to about 24 months after the first treatment session.

9. The method of claim 8, wherein the second treatment session further increases the G-Sn-Pog facial angle of the subject by at least about 1°.

10. The method of claim 1, wherein the injections into the pogonion, mentum, left pre jowl sulcus, and right pre jowl sulcus comprise a serial puncture technique.

11. The method of claim 1, wherein the injections into the pogonion, mentum, left pre jowl sulcus, and right pre jowl sulcus comprise a supraperiosteal injection.

12. The method of claim 1 further comprising injecting a fourth volume of the HA-based dermal filler into a sublabial crease.

13. The method of claim 12, wherein the injection into the sublabial crease comprises a subcutaneous injection.

14. The method of claim 12, wherein a total volume of the HA-based dermal filler injected into the pogonion, mentum, left pre-jowl sulcus, right pre-jowl sulcus, and sublabial crease is no greater than about 4.0 mL.

15. The method of claim 1, wherein each injection volume is no greater than about 2.0 mL.

16. The method of claim 1, wherein the subject has an initial G-Sn-Pog facial angle of about 145° to about 165°.

17. (canceled)

18. (canceled)

19. (canceled)

20. A method of treating chin retrusion in a subject in need thereof, the method comprising:
injecting a first volume of a HA-based dermal filler into a pogonion;
injecting a second volume of the HA-based dermal filler into a mentum; and
injecting a third volume of the HA-based dermal filler into a left pre-jowl sulcus and a right pre-jowl sulcus,
wherein the chin retrusion of the subject is decreased after injection of the HA-based dermal filler into the pogonion, mentum, left pre-jowl sulcus, and right pre-jowl sulcus by at least about 2 mm, and
wherein the HA-based dermal filler composition comprises hyaluronic acid (HA) crosslinked with 1,4-butanediol diglycidyl ether (BDDE);
wherein a HA concentration of the composition is greater than 20 mg/g;
wherein the HA used for crosslinking is made with a mixture of low molecular weight hyaluronic acid and high molecular weight hyaluronic acid; and
wherein the HA used for crosslinking is a mixture containing at least 50% by weight of low molecular weight HA, based on a total weight of the HA.

21. The method of claim 20, wherein the decrease in chin retrusion of the subject is maintained for at least about 3 months.

22. The method of claim 20, wherein the decrease in chin retrusion of the subject is maintained for at least about 12 months.

23. The method of claim 20, wherein the decrease in chin retrusion of the subject is maintained for at least about 18 months.

24. (canceled)

25. (canceled)

26. (canceled)

27. A method of increasing a glabella-subnasale-pogonion (G-Sn-Pog) facial angle in a subject in need thereof, the method comprising:
injecting a first volume of a HA-based dermal filler into a pogonion;
injecting a second volume of the HA-based dermal filler into a mentum; and
injecting a third volume of the HA-based dermal filler into a left pre-jowl sulcus and a right pre-jowl sulcus,
wherein the G-Sn-Pog facial angle of the subject is increased after injection of the HA-based dermal filler into the pogonion, mentum, left pre jowl sulcus, and right pre jowl sulcus by at least about 1°, wherein the increase in the G-Sn-Pog facial angle of the subject is maintained for at least about 18 months, and
wherein the HA-based dermal filler composition comprises hyaluronic acid (HA) crosslinked with 1,4-butanediol diglycidyl ether (BDDE);
wherein a HA concentration of the composition is greater than 20 mg/g;
wherein the HA used for crosslinking is made with a mixture of low molecular weight hyaluronic acid and high molecular weight hyaluronic acid; and
wherein the HA used for crosslinking is a mixture containing at least 50% by weight of low molecular weight HA, based on a total weight of the HA.
